(19) United States
(12) Patent Application Publication
(10) Pub. No.: US 2025/0275533 A1
(43) Pub. Date: Sep. 4, 2025

(54) HYDROXIDES MONOLAYER NANOPLATELET AND METHODS OF PREPARING SAME

(71) Applicant: Aqua Resources Corporation, Fort Walton Beach, FL (US)

(72) Inventor: Orville Lee Maddan, Fort Walton Beach, FL (US)

(21) Appl. No.: 18/917,864

(22) Filed: Oct. 16, 2024

Related U.S. Application Data
(63) Continuation of application No. 17/646,655, filed on Dec. 30, 2021, now abandoned.
Application No. 17/646,655 is a continuation of application No. PCT/US2020/041029, filed on Jul. 7, 2020.
(60) Provisional application No. 63/000,354, filed on Mar. 26, 2020.
(60) Provisional application No. 62/989,511, filed on Mar. 13, 2020.
(60) Provisional application No. 62/874,460, filed on Jul. 15, 2019.

(51) Int. Cl. A01N 25/26; A01N 25/10; A01N 59/16; A01N 59/18; A01N 59/20; A01P 1/00

(52) U.S. Cl. CPC A01N 25/26; A01N 25/10; A01N 59/16; A01N 59/18; A01N 59/20; A01P 1/00

(57) ABSTRACT

Nanoplatelet forms of monolayer metal hydroxides are provided, as well as methods for preparing same. The nanoplatelets are suitable for use in antimicrobial compositions, for pressure treating lumber against wood rot, termites, and fungus, for water treatment for the removal of heavy metal contaminants, for the production of plasmonics devices, for the production of ore, or for the recovery of valuable metals in, e.g., fly ash ponds, mine tailings ponds, or other fluids containing the metal in ionic form. The nanoplatelet forms include copper hydroxide nanoplatelets.

### INCORPORATION BY REFERENCE TO RELATED APPLICATIONS

Any and all priority claims identified in the Application Data Sheet, or any correction thereto, are hereby incorporated by reference under 37 CFR 1.57. This application is a continuation of U.S. application Ser. No. 17/646,655. Filed Dec. 30, 2021, which claims the benefit of PCT Application No. PCT/US2020/041029 filed Jul. 7, 2020, which claims the benefit of U.S. Provisional Application Ser. No. 62/874,460, filed Jul. 15, 2019, U.S. Provisional Application No. 62/989,511, filed Mar. 13, 2020, and U.S. Provisional Application No. 63/000,354, filed Mar. 26, 2020. Each of the aforementioned applications is incorporated by reference herein in its entirety, and each is hereby expressly made a part of this specification.

### FIELD OF THE INVENTION

Nanoplatelet forms of monolayer metal hydroxides are provided, as well as methods for preparing same. The nanoplatelets are suitable for use in antimicrobial compositions, for pressure treating lumber against wood rot, termites, and fungus, for water treatment for the removal of heavy metal contaminants, for the production of plasmonics devices, for the production of ore, or for the recovery of valuable metals in, e.g., fly ash ponds, mine tailings ponds, or other fluids containing the metal in ionic form. The nanoplatelet forms include copper hydroxide nanoplatelets.

### BACKGROUND OF THE INVENTION

Metal hydroxides such as copper hydroxides are useful in a variety of applications. For example, copper hydroxide is useful as a reagent in organic synthesis, in the aquarium industry for its ability to destroy external parasites in fish, as a fungicide and nematicide, as a colorant for ceramic, and as an additive to latex paint to control root growth in potted plants.

### SUMMARY OF THE INVENTION

A method of producing monolayer metal hydroxides of superior properties is desirable. The materials and methods disclosed herein can be employed to prepare such metal hydroxides to form a monolayer nanoplatelet.

The monolayer metal hydroxides of the embodiments are useful in plasmonics-based devices. Plasmonics takes advantage of the coupling of light to charges like electrons in metals, and allows breaking the diffraction limit for the localization of light into subwavelength dimensions enabling strong field enhancements. Plasmonic nanoparticles are discrete metallic particles that have unique optical properties due to their size and shape, and are increasingly being incorporated into commercial products and technologies. These technologies, which span fields ranging from photovoltaics to biological and chemical sensors, take advantage of the extraordinary efficiency of plasmonic nanoparticles at absorbing and scattering light. Additionally, unlike most dyes and pigments, plasmonic nanoparticles have a color that depends on their size and shape and can be tuned to optimize performance for individual applications without changing the chemical composition of the material.

Magnesium hydroxide nanoparticles can be added to a toxic waste stream or volume to advantageously absorb undesirable (e.g., toxic, radioactive, etc.) metal ions by ion or elemental exchange, sequestering the metal in the monolayer metal hydroxides nanoparticles while releasing magnesium ion as a byproduct. The methodology can be used to treat a waste stream such that amounts of radioactive or toxic metals are reduced below safe EPA discharge limits. The methodology is also amenable to use in treatment of large volumes of contaminated water or other waste stream. Magnesium hydroxide nanoparticles are particularly suited for remediation of fly ash and mine tailings, which typically have high levels of undesirable metals. The methodology is also suitable for use in recovering useful, rare, or expensive metals, e.g., in mining operations.

The monolayer metal hydroxides nanoparticles also have antimicrobial properties, and as such can provide remediation of undesirable microbes in addition to metal sequestration. Such nanoparticles can be provided in a form for topical application (e.g., a treatment for athlete's foot, or in a sunscreen, in liquid, gel, or powder form), or can be in a form for ingestion by an organism to provide in vivo antimicrobial properties. For example, copper hydroxide nanoparticles prepared by the methods as described herein are useful for preparing pressure treated lumber with resistance to fungus, mold, and other microbes. Copper hydroxide nanoparticles can be produced that yield superior penetration and distribution of the copper to the lumber's vascular system than is observed for micronized copper as is conventionally employed in the industry. The copper hydroxide nanoparticles provide a single component efficacious treatment for use in preparing pressure treated lumber that is resistant to rot and which exhibits fungicidal properties that extend into years of protection. It is also far less toxic the current products conventionally employed in the industry. The methodology is amenable to large volume production capability and low cost production. The methodology can also be employed to impart a zinc, titanium, or other antimicrobial element as an outer shell of a magnesium hydroxide nanoparticle, so as to deliver the element into the lumber's vasculature. The copper hydroxide monolayer nanoparticles provide a single component efficacious treatment for use in preparing pressure treated lumber that is resistant to rot and is a fungicide that's function extends into years of protection. It is far less toxic than the current products conventionally employed in the industry. The methodology is amenable to large volume production capability and low cost production.

In certain embodiments, the metal hydroxides nanoparticles can advantageously be employed in disinfecting, sanitizing, or antimicrobial compositions. The metal hydroxides nanoparticles have a general antimicrobial effect across infective microorganisms that present a risk to human or veterinary health. Microbes that can be killed or rendered less active or inactive include, but are not limited to viruses, bacteria, protozoa, and fungus. Such microbes can include microbes that are resistant to drugs or other conventional sanitizing agents. Examples include coronaviruses such as the common cold, SARS-associated coronavirus (SARS-CoV, SARS-CoV-2), MERS-associated corona virus (MERS-CoV), and COVID-19, influenza viruses such as Influenza A (H1N1) virus, Zika virus (a member of the virus family Flaviviridae), Marburg virus, Ebola virus, Rabies, HIV, Smallpox, Hantavirus, Dengue virus, Rotavirus, Anthrax, microorganisms that can cause necrotizing fasciitis, e.g., bacteria such as group A beta-hemolytic streptococci (Streptococcus pyogenes), Campylobacter, Salmonella, Staphylococcus aureus, Clostridium perfringens, Clostridium botulinium, Listeria, Eschericha coli, Micobacterium tuberculosis, Klebsiella pneumoniae, Streptococcus pyrogenes, Clostridium difficile, Pseudomonas aeruginosa, Burkholderia cepacia, Acinetobacter baumannii, Neisseria gonorrhoeae, Shigella, Hepatitis A, Hepatitis B, Hepatitis C, Vibrio species, or certain mycotic (fungal) species, e.g., Candida auris, ringworm, Blastomycosis, Coccidioidomycosis, Cryptococcus gatti, Histoplasmosis, Paracoccidioidomycosis, Aspergillosis, Candidiasis, Crypotcoccus neoformans infection, invasive candidiasis, Fusarium, Stachybotrys (e.g., Stachybotrys chartarum), Penicillium, Cladosporum, Mucormycosis, Pneumocystis pneumonia, Talaromycosis, Sporothrix, and the like.

In certain embodiments, the metal hydroxides nanoparticles are employed as an additive to otherwise conventional disinfectant formulations. Depending on the active ingredient used, hand sanitizers can be classified as one of two types: alcohol-based or alcohol-free. Alcohol-based products typically contain between 60 and 95 percent alcohol, usually in the form of ethanol, isopropanol, or n-propanol. At those concentrations, alcohol immediately denatures proteins, effectively neutralizing certain types of microorganisms. Alcohol-free products are generally based on disinfectants, such as benzalkonium chloride (BAC), or on antimicrobial agents, such as triclosan. The activity of disinfectants and antimicrobial agents is both immediate and persistent. Many hand sanitizers also contain emollients (e.g., glycerin) that soothe the skin, thickening agents, and fragrance. The metal hydroxides nanoparticles can be the sole disinfecting or antimicrobial agent present, or can be present in combination with one or more other active agents. The metal hydroxides nanoparticles can be provided in a liquid skin protectant, e.g., an aqueous composition that coats the skin, providing a flexible and breathable barrier to microorganisms and that also provides an antimicrobial effect due to the presence of the metal hydroxides nanoparticles. Such compositions can be provided in a variety of forms suitable for administration, e.g., foam applicator, wipe, spray bottle, and unit dose. The metal hydroxides nanoparticles can be provided in a form of a moistened wipe for cleaning surfaces (e.g., skin, hard surfaces, textiles, or the like). In certain embodiments, textiles or other fibrous materials (e.g., nonwoven fabrics) are coated or impregnated with the metal hydroxides nanoparticles. Examples include surgical masks or other face masks that are dipped in a solution of the metal hydroxides nanoparticles, leaving behind metal hydroxides nanoparticles that provide antimicrobial activity. Filters for masks can be treated in similar fashion, e.g., filter cartridges for full or half coverage face masks, filters including an exhalation valve, filters not including a valve, as can objects such as gowns, drapes, scrubs, coveralls, disposable suits (e.g., Tyvec suits or other such attire), booties, hats, or any item of clothing, gloves, or footwear that can be impregnated with or coated with the metal hydroxides nanoparticles, including nonporous objects, e.g., CPAP masks, examination gloves, durable medical equipment (beds, wheelchairs, etc.). Other materials that can be treated include bedding (sheets, pillows, pillowcases, comforters, mattresses), towels, cleaning sponges, curtains, rugs, carpets, upholstered furniture, and the like. The metal hydroxides nanoparticles can be imparted to the textile or fibrous material in any suitable form, including immersion in a liquid containing the metal hydroxides nanoparticles, a spray or aerosol, a dusting powder, or any other suitable form. In one embodiment, the metal hydroxides nanoparticles are provided as an ingredient in a laundry detergent, a fabric softener, or a fabric rinsing formulation. For cleaning hard surfaces, the metal hydroxides nanoparticles can advantageously be provided in a liquid, foaming, or spray form, e.g., for use in disinfecting hard surfaces in any location where disinfection is desired, e.g., homes, office buildings, airports, schools, prisons, government buildings, retail spaces, restaurants, school, hospitals, medical buildings, or the like. The formulations can find use in those spaces at higher risk of contamination by microorganisms, e.g., home or commercial kitchens, medical equipment, and the like. In one embodiment, the metal hydroxides nanoparticles are provided as an ingredient in a formulation for cleansing the body, e.g., a liquid, gel or foaming hand soap, a soap bar, powder soap, shampoo, body wash, or other similar formulations for cleansing skin, hair, or fur. In one embodiment, the metal hydroxide nanoparticles are provided in a cleanser for dishes or other materials/surfaces that contact food or are contaminated with food residue, e.g., liquid dishwashing soap, dishwasher detergent, formulations for cleansing produce, formulations for cleansing udders, or the like.

In certain embodiments, magnesium hydroxide nanoparticles as described in U.S. Pat. No. 7,892,447 or copper monolayer magnesium hydroxide nanoparticles as described herein are utilized for their antimicrobial properties and ability to kill or render less active viruses, bacteria, protozoa and funguses, e.g., in hand sanitizer wash, “invisible glove” (e.g., coating compositions for use on skin to act as a physical barrier to microbes and other substances), and in respiratory treatments. Such microbes can include coronaviruses, SARS-associated coronavirus, MERS-associated corona virus, and COVID-19, among others. In one embodiment, the magnesium hydroxide nanoparticles can be employed with copper in disinfecting, sanitizing, or antimicrobial compositions and liquid skin protectant. In one embodiment, the magnesium hydroxide nanoplatelets can be employed with zinc in pharmaceutical ethical drugs for preventing or treating respiratory illnesses and diseases, including as an example, COVID-19.

The metal hydroxides nanoparticulates in the forms described herein can be used in conjunction with conventional disinfecting technologies, e.g., ultraviolet light, gamma radiation, ozone, chemical disinfectants (e.g., bleach or hydrogen peroxide, Microban 24), high temperature (e.g., steam or autoclave), physical removal (e.g., washing), encapsulation, and the like.

Hydroxide in nanoparticulate form, e.g., nanoplatelet form, can be purchased from Aqua Resources Corporation or prepared by any suitable method so as to form the core including the method owned Aqua Resources Corporation described in U.S. Pat. Nos. 7,892,447, 7,736,485, 8,822,030, 10,273,163, and 9,604,854. The magnesium hydroxide nanoplatelets so obtained can be subjected to ion exchange to yield copper hydroxide nanoparticles. The magnesium hydroxide nanoplatelets are exposed to copper ion. The copper ion replaces the magnesium ion in the nanoparticles, in part. In certain embodiments, a copper salt (e.g., copper chloride) in solid form or in a form of a copper ion solution is added to an aqueous slurry of magnesium hydroxide nanoplatelets. In other embodiments, magnesium hydroxide nanoparticles in a dry form are added to a solution of copper salt. The amount of copper ion can be selected based on the amount of magnesium ion to be exchanged. For example, less than stoichiometric amounts of copper ion can be provided if partial exchange is desired. Stoichiometric amounts of copper ion can be provided for efficient exchange of the magnesium ion present, or an excess of copper ion can be provided. In certain embodiments, a saturated solution of copper ion is provided, however, in certain embodiments a solution that is not saturated can be provided. Any suitable method for mixing or combining can be employed. The ion exchange can advantageously be conducted at ambient temperatures (e.g., approximately 20° C.), however, in certain other embodiments a liquid mixture of a higher or lower temperature can be employed. The temperature and exposure time can be adjusted such that different degrees of conversion of ion exchange can be achieved. For example, a monolayer of copper hydroxide surrounding a core of magnesium hydroxide can be obtained.

The copper hydroxide monolayer nanoparticles of a small size (e.g., dimensions in the X direction of 30 nm, the Y direction of 30 nm, and the Z direction of 1 nm) up to a larger size (e.g., dimensions in the X direction of 3500 nm, the Y direction of 3500 nm, and the Z direction of 10 nm) can be produced that yield superior penetration and distribution of the copper monolayer nanoparticles to the lumber's vascular system. The nanoplatelets can further comprise a shell encasing a core, wherein the shell comprises copper hydroxide and the core comprises magnesium hydroxide. The nanoplatelets can further comprise a shell encasing a core, wherein the shell comprises zinc hydroxide and the core comprises magnesium hydroxide. The nanoplatelets can further comprise a shell encasing a core, wherein the shell comprises titanium hydroxide and the core comprises magnesium hydroxide. The nanoplatelets can further comprise a shell encasing a core, wherein the shell comprises copper, zinc, titanium hydroxide and the core comprises magnesium hydroxide. This methodology can also be employed to impart a zinc, titanium, or other antimicrobial element as an outer shell of a magnesium hydroxide monolayer nanoparticle, to deliver the element(s) into the lumber's vasculature system.

The nanoplatelets can further comprise a partial shell encasing a core having the molar content of the outer layer of the core to stoichiometric balanced molar content of the ions to produced a shell from about 1% to 99% coverage of the core with individually metal ions or mixed metal ions and the core comprises magnesium hydroxide. In an embodiment of the first aspect, the nanoplatelets comprise individual crystallites.

Hydroxide in nanoparticulate form, e.g., nanoplatelet form, can be purchased from Aqua Resources Corporation or prepared by any suitable method, to form the core of the nanoplatelet, including the method owned by Aqua Resources Corporation and described in U.S. Pat. Nos. 7,892,447, 7,736,485, 8,822,030, 10,273,163, and 9,604,854, the contents of each of which are hereby incorporated by reference in its entirety. These nanoparticulates form the core of monolayer metal hydroxides, which uses a species of a more reactive core hydroxide. The outermost layer of the core hydroxides are replaced by metal ions mixed in water or other suitable fluid for ion exchange, the suitable fluid containing a dissolved metal ion species not as reactive and not the same as the core metal species to form the monolayer hydroxides shell.

Nanoplatelets of metal hydroxide are mixed in to the water column or other suitable fluid as a precursor to form the core of a monolayer nanoplatelet, with metal salts or other ions provided as supply sources that are dissolved in to the water column or other suitable fluid, to supply the ions to self assemble by ion exchange the monolayer shell, thereby creating a metal hydroxide monolayer nanoplatelets.

The monolayer shell is formed by ion exchange from the hydroxide core with a less reactive metal ion from the water column or other suitable fluid there by reducing that shell species concentration in the fluid column and increasing core ion content of the fluid column. Metal hydroxide monolayer nanoplatelets with the core comprised of a more reactive metal hydroxide, and a less reactive metal hydroxide shell, wherein the shell is not same Metal hydroxide, having platelet diameter of from about 30 nm to about 3500 nm and thickness of from about 1 nm to about 400 nm., comprising individual crystallites.

Methods and methodologies used to produce monolayer nanoplatelets are provided. The base materials used to make the process feedstock include, for example, magnesium hydroxide nanoplatelets for the core and copper chloride or another soluble copper salt. Suitable shell materials include commercially available bulk forms of copper chloride. While copper chloride is particularly preferred, other sources of soluble copper ion can also be employed, for example, other copper halides such as copper bromide and copper iodide, copper nitrate, and copper sulfide. Any suitable nanoparticulate form of magnesium hydroxide can be used as a starting core material; however, magnesium hydroxide as prepared according to the method described herein can advantageously be employed.

As used herein, the term “metal hydroxide” is employed to refer to a metal hydroxide, a metal oxide, or mixtures thereof. Metal oxides and hydroxides more reactive form the core, and are less reactive than the shell ions. The core metal oxides and hydroxides include but are not limited to scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, ununbium lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, and lawrencium. mixed oxides and hydroxides of the foregoing metals, mixed metal oxides and/or hydroxides, and other combinations thereof.

The shell is formed from ions of less reactive metal ions than the core of metal oxides and/or hydroxides to form the monolayer shell of metal oxides and/or hydroxides by ion exchange. Such metals include but are not limited to ions of scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, ununbium lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, and lawrencium and other combinations thereof.

A method of producing metal hydroxides of superior properties as described above is therefore desirable. The materials and methods disclosed herein can be employed to prepare such metal hydroxides, e.g., copper hydroxides, in nanoplatelet and other forms. The metal hydroxide is preferably in nanoparticulate form (e.g., nanoplatelet form), although other configurations can also be prepared.

Accordingly, in a first aspect (independently combinable in whole or in part with any other aspect or embodiment), a nanoplatelet is provided having a metal hydroxide monolayer, produced by: mixing a nanoplatelet of magnesium hydroxide into a water column as a precursor to form a core of a monolayer nanoplatelet; dissolving metal salts or metal ion sources to supply other metal ions that are dissolved into the water of the water column to supply the other metal ions to self assemble by ion exchange to yield a monolayer shell, thereby creating a metal hydroxide monolayer nanoplatelet.

In an embodiment of the first aspect (independently combinable in whole or in part with any other aspect or embodiment), the monolayer shell is formed by ion exchange from the magnesium hydroxide core with a less reactive metal ion from the water column, thereby reducing that shell species concentration in the water column and increasing magnesium ion content of the water column.

In an embodiment of the first aspect (independently combinable in whole or in part with any other aspect or embodiment), the nanoplatelet is a metal hydroxide monolayer nanoplatelet with the core comprised of magnesium hydroxide, and a metal hydroxide shell, wherein the shell does not comprise magnesium, the nanoplatelets having a platelet diameter of from about 30 nm to about 3500 nm, a thickness of from about 1 nm to about 400 nm and an aspect ratio of from 15 to 75.

In an embodiment of the first aspect (independently combinable in whole or in part with any other aspect or embodiment), the nanoplatelet is an individual crystallite.

In an embodiment of the first aspect (independently combinable in whole or in part with any other aspect or embodiment), the nanoplatelet comprises a shell encasing a core, wherein the shell comprises a transition metal ions selected from the group consisting of scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, and ununbium, individually or mixtures thereof, and the core comprises magnesium hydroxide.

In an embodiment of the first aspect (independently combinable in whole or in part with any other aspect or embodiment), the nanoplatelet comprises a shell encasing a core, wherein the shell comprises a lanthanide series elements, ions selected from the group consisting of lanthanum, cerium, praseodymium, neodymium, promethium, samarium, europium, gadolinium, terbium, dysprosium, holmium, erbium, thulium, ytterbium, and lutetium individually or mixtures thereof, and the core comprises magnesium hydroxide.

In an embodiment of the first aspect (independently combinable in whole or in part with any other aspect or embodiment), the nanoplatelet comprises a shell encasing a core, wherein the shell comprises of a rare earth is an actinide series element, ions selected from the group consisting of actinium, thorium, protactinium, uranium, neptunium, plutonium, americium, curium, berkelium, californium, einsteinium, fermium, mendelevium, nobelium, and lawrencium, individually or mixtures thereof, and the core comprises magnesium hydroxide.

In an embodiment of the first aspect (independently combinable in whole or in part with any other aspect or embodiment), the nanoplatelet has antimicrobial properties and comprises a shell encasing a core, wherein the shell comprises metal ions selected from the group consisting of titanium, zinc, silver and copper, individually or mixtures thereof, and the core comprises magnesium hydroxide.

In an embodiment of the first aspect (independently combinable in whole or in part with any other aspect or embodiment), the nanoplatelet has a molar content of the outer layer of the core to a stoichiometric balanced molar content of the ions to produce a shell providing from about 1% to 99% coverage of the core with individual metal ions or mixed metal ions.

In a second aspect (independently combinable in whole or in part with any other aspect or embodiment), a nanoplatelet is provided comprising a metal hydroxide monolayer shell, prepared by: mixing an insoluble metal hydroxide more active than shell metal ions into a water column containing the shell metal ions as a precursor to forming a core of a monolayer nanoplatelet, wherein the shell metal ions self assemble by ion exchange a monolayer shell on the core, thereby creating a metal hydroxide monolayer nanoplatelet concentrating the shell metal ions in the monolayer shell as an ore to be reduced to a pure element.

In an embodiment of the second aspect (independently combinable in whole or in part with any other aspect or embodiment), the nanoplatelet is a metal hydroxide monolayer nanoplatelet, wherein the core does not comprise magnesium hydroxide, the nanoplatelets comprising a metal hydroxide shell, wherein the shell does not comprise magnesium hydroxide, the nanoplatelets having a platelet diameter of from about 30 nm to about 3500 nm, a thickness of from about 1 nm to about 400 nm, and an aspect ratio of 15 to 75

In an embodiment of the second aspect (independently combinable in whole or in part with any other aspect or embodiment), the core comprises metal hydroxide selected from the group consisting of scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, ununbium lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, and lawrencium, individually or mixtures thereof.

In an embodiment of the second aspect (independently combinable in whole or in part with any other aspect or embodiment), the shell comprises metal ions selected from the group consisting of scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, ununbium lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, and lawrencium, individually or mixtures thereof.

In an embodiment of the second aspect (independently combinable in whole or in part with any other aspect or embodiment), the nanoplatelet is an individual crystallite.

In a third aspect (independently combinable in whole or in part with any other aspect or embodiment), a nanoparticle is provided comprising a first metal hydroxide shell surrounding a second metal hydroxide core, wherein the second metal is more labile than the first metal.

In an embodiment of the third aspect (independently combinable in whole or in part with any other aspect or embodiment), the nanoplatelet has a dimension in the X axis of from about 30 nm to about 3500 nm, a dimension in the Y axis of about 30 nm to about 3500 nm, a dimension in the Z axis of from 1 nm to 400 nm, and an aspect ratio of 15 to 75.

The nanoparticle of Claim 15, wherein the nanoparticle comprises a copper hydroxide shell and a magnesium hydroxide core.

In an embodiment of the third aspect (independently combinable in whole or in part with any other aspect or embodiment) the shell comprises a monolayer of the first metal hydroxide.

In an embodiment of the third aspect (independently combinable in whole or in part with any other aspect or embodiment), the nanoplatelet is an individual nanoplatelet crystallite.

In an embodiment of the third aspect (independently combinable in whole or in part with any other aspect or embodiment), the nanoplatelet is for use in removing metal ions from water containing fly ash, the nanoparticle having a dimension in the X axis of from about 30 nm to about 100 nm, a dimension in the Y axis of about 30 nm to about 100 nm, a dimension in the Z axis of from 1 nm to 10 nm, and an aspect ratio of 15 to 75.

In an embodiment of the third aspect (independently combinable in whole or in part with any other aspect or embodiment), the nanoplatelet is for use in removing metal ions from water containing fly ash, the nanoparticle having a dimension in the X axis of less than 150 nm, a dimension in the Y axis of less than 150 nm, a dimension in the Z axis of from 1 nm to 10 nm, and an aspect ratio of 15 to 75.

In an embodiment of the third aspect (independently combinable in whole or in part with any other aspect or embodiment), the nanoplatelet is for use as an in vivo antibacterial agent, the nanoparticle having a dimension in the X axis of 150 nm to 3500 nm, a dimension in the Y axis of 150 nm to 3500 nm, a dimension in the Z axis of from 1 nm to 10 nm, and an aspect ratio of 15 to 75.

In an embodiment of the third aspect (independently combinable in whole or in part with any other aspect or embodiment), the nanoplatelet is for use as an antibacterial agent for topical application to human skin, the nanoparticle having a dimension in the X axis of 150 nm to 3500 nm, a dimension in the Y axis of 150 nm to 3500 nm, a dimension in the Z axis of from 1 nm to 10 nm, and an aspect ratio of 15 to 75.

In an embodiment of the third aspect (independently combinable in whole or in part with any other aspect or embodiment), the nanoplatelet is for use as an antibacterial agent for topical application to human skin, the nanoparticle having a dimension in the X axis of 150 nm to 3500 nm, a dimension in the Y axis of 150 nm to 3500 nm, a dimension in the Z axis of from 1 nm to 10 nm, and an aspect ratio of 15 to 75.

In an embodiment of the third aspect (independently combinable in whole or in part with any other aspect or embodiment), the nanoplatelet is for use in pressure treating lumber fabricated from a species of tree, wherein the dimension in the X axis, the dimension in the Y axis, and the dimension in the Z axis of the nanoparticle are selected so as to permit the nanoparticle to penetrate into the vasculature of the species of tree, optionally into the small capillaries of the species of tree.

In a fourth aspect (independently combinable in whole or in part with any other aspect or embodiment), a nanoplatelet is provided having a dimension in the X axis of from about 30 nm to about 3500 nm, a dimension in the Y axis of about 30 nm to about 3500 nm, a dimension in the Z axis of from 1 nm to 400 nm, and an aspect ratio of 15 to 75, wherein the nanoplatelet comprises copper hydroxide.

In an embodiment of the fourth aspect (independently combinable in whole or in part with any other aspect or embodiment), the nanoplatelet consists essentially of copper hydroxide.

In an embodiment of the fourth aspect (independently combinable in whole or in part with any other aspect or embodiment), the nanoplatelet further comprises magnesium hydroxide.

In an embodiment of the fourth aspect (independently combinable in whole or in part with any other aspect or embodiment), the nanoplatelet further comprises a shell encasing a core, wherein the shell comprises copper hydroxide and the core comprises magnesium hydroxide.

In an embodiment of the fourth aspect (independently combinable in whole or in part with any other aspect or embodiment), the shell comprises a monolayer of copper hydroxide.

In an embodiment of the fourth aspect (independently combinable in whole or in part with any other aspect or embodiment), the nanoplatelet is an individual crystallite.

In a fifth aspect (independently combinable in whole or in part with any other aspect or embodiment), a nanoplatelet is provided having a dimension in the X axis of from about 30 nm to about 3500 nm, a dimension in the Y axis of about 30 nm to about 3500 nm, a dimension in the Z axis of from 1 nm to 400 nm, and an aspect ratio of 15 to 75, wherein the nanoplatelet comprises a magnesium hydroxide core and a metal hydroxide or rare earth hydroxide shell, wherein the metal or the rare earth is not magnesium.

In an embodiment of the fifth aspect (independently combinable in whole or in part with any other aspect or embodiment), the nanoplatelet further comprises the metal is a transition metal selected from the group consisting of scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, gold, mercury, rutherfordium, dubnium, seaborgium, bohrium, hassium, meitnerium, ununnilium, unununium, and ununbium.

In an embodiment of the fifth aspect (independently combinable in whole or in part with any other aspect or embodiment), the rare earth is a lanthanide series element selected from the group consisting of lanthanum, cerium, praseodymium, neodymium, promethium, samarium, europium, gandolinium, terbium, dysoprosium, holmium, erbium, thulium, ytterbium, and lutetium.

In an embodiment of the fifth aspect (independently combinable in whole or in part with any other aspect or embodiment), the rare earth is an actinide series element selected from the group consisting of actinium, thorium, protactinium, uranium, neptumium, plutonium, americium, curium, berkelium, californium, einsteinium, fermium, mendelevium, nobelium, and lawrencium.

In an embodiment of the fifth aspect (independently combinable in whole or in part with any other aspect or embodiment), the shell comprises a monolayer of the metal hydroxide or rare earth hydroxide.

In an embodiment of the fifth aspect (independently combinable in whole or in part with any other aspect or embodiment), the nanoplatelet is an individual crystallite.

In a sixth aspect (independently combinable in whole or in part with any other aspect or embodiment), a nanoplatelet is provided having a dimension in the X axis of from about 30 nm to about 3500 nm, a dimension in the Y axis of about 30 nm to about 3500 nm, a dimension in the Z axis of from 1 nm to 400 nm, and an aspect ratio of 15 to 75, wherein the nanoplatelet comprises zinc hydroxide or titanium hydroxide.

In an embodiment of the sixth aspect (independently combinable in whole or in part with any other aspect or embodiment), the nanoplatelet consists essentially of copper hydroxide.

In an embodiment of the sixth aspect (independently combinable in whole or in part with any other aspect or embodiment), the nanoplatelet further comprises magnesium hydroxide.

In an embodiment of the sixth aspect (independently combinable in whole or in part with any other aspect or embodiment), the nanoplatelet comprises a shell encasing a core, wherein the shell comprises copper hydroxide and the core comprises magnesium hydroxide.

In an embodiment of the sixth aspect (independently combinable in whole or in part with any other aspect or embodiment), the shell comprises a monolayer of copper hydroxide.

In an embodiment of the sixth aspect (independently combinable in whole or in part with any other aspect or embodiment), the nanoplatelet is an individual crystallite.

In a seventh aspect (independently combinable in whole or in part with any other aspect or embodiment), a method is provided of forming a nanoparticle comprising: exposing a first metal hydroxide nanoparticle to a second metal ion, whereby a first metal ion of the nanoparticle is replaced by the second metal ion, yielding a nanoparticle comprising a second metal hydroxide shell surrounding a first metal hydroxide core.

In an embodiment of the seventh aspect (independently combinable in whole or in part with any other aspect or embodiment), the second metal is copper and wherein the first metal is magnesium.

In an embodiment of the seventh aspect (independently combinable in whole or in part with any other aspect or embodiment), only a portion of magnesium is replaced, such that the resulting nanoparticle comprises copper hydroxide and magnesium hydroxide.

In an embodiment of the seventh aspect (independently combinable in whole or in part with any other aspect or embodiment), the resulting nanoparticle comprises a copper hydroxide shell encasing a magnesium hydroxide core.

In an embodiment of the seventh aspect (independently combinable in whole or in part with any other aspect or embodiment), the shell comprises a monolayer of copper hydroxide.

In an embodiment of the seventh aspect (independently combinable in whole or in part with any other aspect or embodiment), the nanoparticle is an individual crystallite.

In an embodiment of the seventh aspect (independently combinable in whole or in part with any other aspect or embodiment), the nanoparticle has a dimension in the X axis of from about 30 nm to about 3500 nm, a dimension in the Y axis of about 30 nm to about 3500 nm, a dimension in the Z axis of from 1 nm to 400 nm, and an aspect ratio of 15 to 75.

In an embodiment of the eighth aspect (independently combinable in whole or in part with any other aspect or embodiment), a method is provided of forming a nanoparticle comprising copper hydroxide, comprising: exposing a magnesium hydroxide nanoparticle to a copper ion, whereby a magnesium ion is replaced by the copper ion, yielding a nanoparticle comprising copper hydroxide.

In an embodiment of the eighth aspect (independently combinable in whole or in part with any other aspect or embodiment), the nanoparticle comprising copper hydroxide consists essentially of copper hydroxide.

In an embodiment of the eighth aspect (independently combinable in whole or in part with any other aspect or embodiment), only a portion of magnesium ion is replaced by copper ion, such that the nanoparticle comprising copper hydroxide further comprises magnesium hydroxide.

In an embodiment of the eighth aspect (independently combinable in whole or in part with any other aspect or embodiment), the nanoparticle comprising copper hydroxide comprises a shell encasing a core, wherein the shell comprises copper hydroxide and the core comprises magnesium hydroxide.

In an embodiment of the eighth aspect (independently combinable in whole or in part with any other aspect or embodiment), the shell comprises a monolayer of copper hydroxide.

In an embodiment of the eighth aspect (independently combinable in whole or in part with any other aspect or embodiment), the nanoparticle comprising copper hydroxide is an individual crystallite.

In an embodiment of the eighth aspect (independently combinable in whole or in part with any other aspect or embodiment), the nanoparticle has a dimension in the X axis of from about 30 nm to about 3500 nm, a dimension in the Y axis of about 30 nm to about 3500 nm, a dimension in the Z axis of from 1 nm to 400 nm, and an aspect ratio of 15 to 75.

In a ninth aspect (independently combinable in whole or in part with any other aspect or embodiment), a method is provided of removing ions of a metal other than magnesium from a substance or removing a rare earth from a substance, comprising: exposing the substance to magnesium hydroxide nanoparticles, whereby magnesium ions are replaced by the metal ions or the rare earth ions, yielding a nanoparticle comprising a hydroxide of the metal or a hydroxide of the rare earth; and sequestering the nanoparticle comprising the hydroxide of the metal or the hydroxide of the rare earth from the substance.

In an embodiment of the ninth aspect (independently combinable in whole or in part with any other aspect or embodiment), the substance is water that has been exposed to a fly ash or mine tailings.

In an embodiment of the ninth aspect (independently combinable in whole or in part with any other aspect or embodiment), the method is a method of mining the metal or the rare earth.

In a tenth aspect (independently combinable in whole or in part with any other aspect or embodiment), a method is provided of pressure treating lumber, comprising: exposing the lumber to nanoplatelets having a dimension in the X axis of from about 30 nm to about 3500 nm, a dimension in the Y axis of about 30 nm to about 3500 nm, a dimension in the Z axis of from 1 nm to 400 nm, and an aspect ratio of 15 to 75, wherein the nanoplatelets comprise copper hydroxide, such that the nanoplatelets penetrate into a vasculature of the lumber, whereby a resistance to a destructive organism is imparted to the lumber.

In an embodiment of the tenth aspect (independently combinable in whole or in part with any other aspect or embodiment), the organism is selected from the group consisting of wood rot, termites, and fungus.

In an embodiment of the tenth aspect (independently combinable in whole or in part with any other aspect or embodiment), the dimension in the X axis, the dimension in the Y axis, and the dimension in the Z axis of each of the nanoplatelets is selected so as to permit the nanoplatelets to penetrate into the vasculature of the lumber, optionally into small capillaries of the lumber.

In an eleventh aspect (independently combinable in whole or in part with any other aspect or embodiment), a method is provided of imparting antimicrobial properties, comprising: exposing a surface to nanoplatelets having a dimension in the X axis of from about 30 nm to about 3500 nm, a dimension in the Y axis of about 30 nm to about 3500 nm, a dimension in the Z axis of from 1 nm to 400 nm, and an aspect ratio of 15 to 75.

In an embodiment of the eleventh aspect (independently combinable in whole or in part with any other aspect or embodiment), the nanoplatelets comprise copper hydroxide.

In an embodiment of the eleventh aspect (independently combinable in whole or in part with any other aspect or embodiment), the nanoplatelets each have a dimension in the X axis of 150 nm to 3500 nm, a dimension in the Y axis of 150 nm to 3500 nm, a dimension in the Z axis of from 1 nm to 10 nm, and an aspect ratio of 15 to 75.

In an embodiment of the eleventh aspect (independently combinable in whole or in part with any other aspect or embodiment), the nanoplatelets each have a dimension in the X axis of 150 nm to 3500 nm, a dimension in the Y axis of 150 nm to 3500 nm, a dimension in the Z axis of from 1 nm to 10 nm, and an aspect ratio of 15 to 75.

In an embodiment of the eleventh aspect (independently combinable in whole or in part with any other aspect or embodiment), the surface is a skin surface, wherein the nanoplatelets are provided in a form of a topical composition, optionally an athletes foot treatment or a sunscreen.

In an embodiment of the eleventh aspect (independently combinable in whole or in part with any other aspect or embodiment), the nanoplatelets are consumed by an organism, optionally a fish or a mammal, optionally a human, a cow, a pig, a chicken, a duck, a turkey, a goat, or a sheep.

### BRIEF DESCRIPTION OF THE DRAWINGS

The detailed description is described with reference to the accompanying figures.

FIG. 1 is a micrograph showing magnesium hydroxide nanoplatelets.

### DETAILED DESCRIPTION OF THE PREFERRED EMBODIMENT

Methods and methodologies used to produce nanoplatelets and other forms of metal hydroxides, e.g., copper hydroxide, are provided. The base materials used to make the process feedstock include, for example, magnesium hydroxide nanoplatelets and copper chloride or another soluble copper salt. Suitable base materials include commercially available bulk forms of copper chloride. While copper chloride is particularly preferred, other sources of soluble copper ion can also be employed, for example, other copper halides such as copper bromide and copper iodide, copper nitrate, and copper sulfide. Any suitable nanoparticulate form of magnesium hydroxide can be used as a starting material; however, magnesium hydroxide as prepared according to the method described herein can advantageously be employed. As used herein, the term “metal (hydr)oxide” is employed to refer to a metal hydroxide, a metal oxide, or mixtures thereof. Metal oxides and hydroxides include, but are not limited to, MgO, SrO, BaO, CaO, TiO₂, ZrO₂, FeO, V₂O₃, V₂O₅, Mn₂O₃, Fe₂O₃, NiO, Ni₂O₃, CuO, Al₂O₃, SiO₂, ZnO, Ag₂O, [Ce(NO₃)₃—Cu(NO₃)₂] TiO₂, Mg(OH)₂, Ca(OH)₂, Al(OH)₃, Sr(OH)₂, Ba(OH)₂, Fe(OH)₃, Cu(OH)₃, Cu(OH)₂, CuOH, Ni(OH)₂, Co(OH)₂, Zn(OH)₂, AgOH, mixed oxides and hydroxides of the foregoing metals, mixed metal oxides and/or hydroxides, and other combinations thereof.

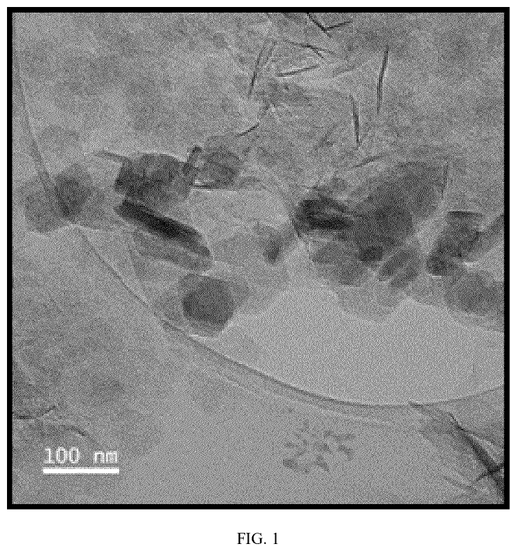

Hydroxide in nanoparticulate form, e.g., nanoplatelet form, can be purchased from Aqua Resources Corporation, or prepared by any suitable method. For example, magnesium hydroxide in nanoparticulate form, e.g., nanoplatelet form, can be prepared by the methods described in U.S. Pat. Nos. 7,892,447, 7,736,485, 8,822,030, 10,273,163, and 9,604,854, the contents of each of which are hereby incorporated by reference in its entirety. Such nanoparticulates, as shown in FIG. 1, form the core of the metal hydroxide monolayer nanoparticulates prepared according to the methods herein. The more reactive core hydroxides, e.g., magnesium hydroxide, are preferably employed in preparing the metal hydroxide monolayer nanoparticulates; however, any metal hydroxide core can be used provided the metal of the core is preferentially replaced in the nanoparticle by the metal ion of interest (different from the metal ion of the core) for forming a monolayer. The outer most layer of the core metal hydroxide hydroxide(s) is exposed to water containing a dissolved metal ion species of interest for ion exchange. While not wishing to be bound by theory, it is believed that the dissolved ion species, which is different from the core species, is not as reactive as the core species in its propensity to form a monolayer hydroxide shell.

### Preparation of Metal Hydroxide Core

As discussed above, the method for preparing metal monolayer nanoparticles starts with a nanoparticulate core. Any metal hydroxide nanoparticulates, prepared by any method, can be employed. In the methods described in U.S. Pat. Nos. 7,892,447, 7,736,485, 8,822,030, 10,273,163, and 9,604,854, for example, magnesium chloride and sodium chloride are mixed in a feedstock tank, with reverse osmosis (RO) water as a solvent to yield an ionic and gelatinous fluid. RO water is typically prepared by taking regular tap water, running it through a water softener, and then running the softened water through a reverse osmosis system. The purity of the RO water is similar to that of de-ionized (DI) water, but is considerably cheaper to produce. While RO water is generally preferred as a solvent due to its reduced costs, DI water, or water of similar purity can be employed as well. In certain embodiments, water of lesser purity (e.g., tap water) can be employed in the preparation of the metal (hydr)oxides of preferred embodiments. The sodium chloride brings the electrolyte content of the water up so as to reduce its electrical resistance, thereby reducing electrical costs for the production of the metal hydroxide nanoplatelets or other metal oxide or hydroxide forms. While it is generally preferred to employ sodium chloride, other suitable electrolytes can also be employed, alone or in combination. Common electrolytes include ions such as sodium (Na⁺), lithium (Li⁺), potassium (K⁺), calcium (Ca²⁺), magnesium (Mg²⁺), chloride (Cl⁻), fluoride (F⁻), bromide (Br⁻), and the like.

The quantity of magnesium chloride and sodium chloride dissolved in the water is selected, along with other process conditions, so as to yield magnesium hydroxide particles of desired properties (e.g., particle size and/or morphology). In preferred embodiments, magnesium chloride is added to the RO water so as to yield a brine containing from about 300 ppm (or less) to about 100,000 ppm (or more) of magnesium ions, preferably about 1,500 ppm of magnesium ions. Sodium chloride is added to the RO water so as to yield a brine containing from about 5,000 ppm to about 200,000 ppm Cl⁻, preferably about 43,000 ppm Cl⁻.

For example, about 8 fluid ounces (237 mg) of concentrated muriatic acid (hydrochloric acid) is added to every 400 gallons (1,514 liters) of RO water that has been blended with magnesium chloride and sodium chloride to achieve a pH of approximately 4.5. At that pH level, any carbonates that are present in the salts are dissolved into the water. While a pH of 4.5 is particularly preferred, any suitable pH that will dissolve carbonate can also be employed. The dissolved carbonates are then removed by conversion into CO₂ gas which is degassed from the system, thereby minimizing carbonates solids forming in the precipitation operation. While muriatic acid is preferred for use in adjusting pH, other suitable acids can also be employed (e.g., hydrobromic acid, sulfuric acid, and the like). Depending upon the carbonate content (or lack thereof) of the feedstock materials, it can not be necessary to adjust the pH. Alternatively, in certain embodiments, the presence of carbonates solids in the precipitation operation is tolerable, and thus no special procedures for removing them are performed.

After the system is degassed of carbon dioxide (CO₂), the magnesium ion-containing feedstock is circulated through a heating system. It is preferred to operate the system at a temperature of from about 40° F. to about 200° F. (about 4° C. to about 93° C.), preferably from about 50° F. to about 190° F. (about 10° C. to about 88° C.), more preferably from about 60° F. to about 160° F. (about 16° C. to about 71° C.), even more preferably about 110° F. (about 43° C.). In some embodiments, the system can be operated at any suitable temperature from about the freezing point of the feedstock to about the boiling point of the feedstock.

The feedstock is then sent to an electrolyzer divided into three compartments: an anode compartment; a cathode compartment; and a center fluid separator compartment between the anode and cathode compartments that separates fluids of the anodic compartment from the fluids of the cathodic compartment. While this particular electrolyzer configuration is generally preferred, other suitable configurations can also be employed. Other suitable electrolyzer configurations are described in U.S. Pat. Nos. 7,048,843, 6,235,185, 6,375,825, 5,660,709, 5,496,454, and 5,785,833, the disclosures of each of which are herein incorporated by reference in their entirety. Still other suitable electrolyzer configurations are outlined in U.S. Patent Publication No. 2003/0082095, U.S. Patent Publication No. 2004/0197255, U.S. Patent Publication No. 2007/0113779, and U.S. Patent Publication No. 2004/0108220, the disclosures of each of which are herein incorporated by reference in their entirety.

In one exemplary configuration, within the anode compartment is an anode electrode that is constructed out of titanium and coated with iridium oxide, and is in the form of a mesh with ¼-inch holes throughout (manufactured by Uhdenora S.p.A. of Milan, Italy; UHDE BM-2.7, anode compartment half shell including the anode electrode). Other electrodes and electrode configurations can also be employed (e.g., other metals such as platinum group metals (platinum or ruthenium, or oxides thereof), or plate or bar shaped electrodes instead of mesh). The compartment can operate from 0.1 to 3.75 amps per square inch; however, in certain embodiments it can be acceptable or even desirable to operate at lower or higher amperages. The electrical circuit for the anode compartment chamber can operate or be operated in either a series or parallel configuration, as is commonly employed in the chlor-alkali industry. NaCl in the anode compartment is split into chlorine gas and sodium ion (Na⁺). The sodium ion travels through an ion selective membrane (manufactured by The Dow Chemical Company of Midland, Michigan) into the center compartment. Any suitable membrane can be employed that is permeable to sodium ions but resists the flow of water there through. Examples of ion selective membranes include glass membranes (e.g., silicates of chalcogenides), crystalline membranes (e.g., fluoride selective electrodes based on LaF₃crystals), and ion exchange resin membranes (anion exchange, cation exchange, and mixed ion exchange membranes such as those prepared from polyvinyl, polystyrene, polyethylene, polyesters, epoxies, and silicones).

In an exemplary embodiment, the cathode electrode, which is in the cathode chamber, can constructed out of a nickel alloy in a perforated form to create many flux lines there through (manufactured by Uhdenora S.p.A. of Milan, Italy; UHDE BM-2.7, cathode half shell including electrode). Alternatively, the cathode electrode can be constructed of #316 stainless steel. Other electrode configurations and materials can also be suitable for use (e.g., electrode materials and configurations as described above with reference to the anode electrode), with process conditions adjusted accordingly. The cathode compartment can operate with a sodium hydroxide solution up to about 50% by weight or can operate with a NaCl solution or other electrolyte solution. The cathode compartment can also operate at 3.75 amps per square inch or less. The electrical circuit for the cathode compartment chamber can operate or be operated in either a series or parallel configuration. Water is split in the cathode chamber to yield hydrogen gas and hydroxyl ions. An ion selective membrane rests on the cathode electrode and faces the center fluid separator compartment, allowing hydroxyl ions to pass there through.

The fluid within the anodic compartment is preferably at a pH of about 1, and the fluid within the cathode compartment is preferably at a pH above 8.5. Electricity can flow freely through the center fluid separator compartment, but hydrophobic ion selective membranes restrict the movement of water into it, thereby allowing the cathode compartment and anode compartment to contain their own separate fluids. The center compartment includes an inlet and an outlet and is situated between the two ion selective membranes (manufactured by The Dow Chemical Company of Midland, Michigan). The center chamber operates with a positive pressure to keep each of the membranes in place. Suitable membranes include electrodeionization membranes such as those sold under the trademark OMEXELL™ by The Dow Chemical Company.

The ion selective membranes selectively allow ions to pass into the center fluid separator compartment where magnesium hydroxide precipitation takes place, and from which magnesium hydroxide nanoplatelets are harvested. This process occurs as follows. A sodium ion passes through the anode ion selective membrane into the center compartment, and a hydroxyl ion passes through the cathode ion selective membrane into the center compartment. Magnesium ions from the magnesium chloride in the center compartment react with hydroxyl ions to form solid magnesium hydroxide leaving a free chlorine ion. The sodium ion from the anode compartment reacts with the free chlorine ion from the magnesium chloride in the center compartment to form sodium chloride.

Prior to initiating metal hydroxide precipitation in the electrolyzer, RO water is heated in a tank to a temperature of about 120° F. Sodium chloride is added to the RO water until a Cl⁻ concentration of about 30,000 parts per million to about 200,000 parts per million is reached, yielding a very conductive solution. In a preferred embodiment, sodium chloride is added to the RO water until a Cl concentration of about 75,000 parts per million is reached. The RO water with added sodium chloride at the elevated temperature is pumped into the center chamber to fill it completely. The cathode and anode compartments are then filled with their respective fluids. Next, the current in the electrolyzer is brought up to the predetermined level, preferably levels as described above. Current in the electrolyzer can be brought to a current of from about 4.00 Amps per square inch to about 0.10 Amps per square inch. In certain embodiment, it may be desirable to achieve a current higher than 4.00 Amps per square inch or lower than 0.10 Amps per square inch. In a preferred embodiment, the current is brought to about 0.75 Amps per square inch. The current generates hydroxyl (OH⁻) ions in the cathode chamber by splitting water, thereby driving the pH up.

When a predetermined pH, such as a pH of about 11, is reached throughout the fluid, the magnesium-containing feedstock is added to the center fluid separator compartment. When the magnesium-containing feedstock is added to the high pH RO water with added sodium chloride, the magnesium ions are attracted to the electrode flux grid line, where they react with hydroxyls to yield magnesium hydroxide. After nucleation, magnesium hydroxide adds to the nucleus in a flat plane along the flux lines, such that in the remaining crystal growth, magnesium hydroxides attach around the border of the nucleation crystal in conformity with the flux lines, yielding crystalline nanoplatelets in the center compartment. By adjusting selected variables, the particle size and morphology of the magnesium hydroxide can be controlled. The residence time of feedstock flow through the center compartment can be adjusted to set the particle size (with faster flow rates resulting in smaller particle size). Feedstock residence times of from about 0.1 minute or less to about 10 minutes or more are generally preferred. The quality of flux line by the energy passing between the opposing compartments and temperature can be adjusted to control the speed of the reaction. By adjusting these parameters, magnesium hydroxide platelets of uniform size can be produced. Tight size distributions can be obtained for particles having an average platelet size of 3.5 microns in the X/Y dimension and 100 nm in the Z dimension down to particles having an average platelet size of 30 nm in the X/Y dimension and 2.5 nm in the Z dimension. Generally, the faster the nanoplatelets are harvested, as long as a pH above the precipitation point is maintained for the metal being produced, the smaller the resulting nanoplatelets. The preferred dimensions will depend upon the application and the system will be adjusted accordingly.

Sodium chloride (NaCl) is converted to chlorine (Cl₂ gas) in the anode compartment, and the resulting sodium ion migrates to the cathode compartment. As discussed above, in the cathode compartment water is split to release hydrogen gas (H₂ gas), leaving a hydroxyl ion which combines with a magnesium ion from the metal chloride to form magnesium hydroxide. Chloride ion combines with the sodium ion in the cathode compartment to form sodium chloride.

The electrolyzer incorporates a pipe that allows elemental hydrogen gas generated during water splitting to leave the cathode compartment. Another pipe in the anode compartment allows elemental chlorine gas produced to leave. In the production of magnesium hydroxide, approximately 6.34 cubic feet of hydrogen gas weighing 0.07 pounds is generated for every pound of magnesium hydroxide that is produced, and approximately 6 cubic feet of chlorine gas weighing 1.2 pounds is generated for every pound of magnesium hydroxide produced. The hydrogen and/or chlorine gas can be disposed of, or captured for use as feedstocks in other processes. In preferred embodiments, the chlorine gas can be employed to produce sodium hydrochloric bleach at a 15% density. While a pipe is a particularly preferred component for venting gas, other components can also be employed (e.g., a passageway, a gas permeable sheet, or the like).

The nanoplatelet-containing fluid is removed to a catch basin, and then to a centrifuge where magnesium hydroxide is separated from the supernatant containing ions in water. The supernatant is recycled back into the feedstock system so as to recover magnesium ions, sodium ions, and chlorine ions. After the centrifuge discharges the magnesium hydroxide solids in the form of a gel, the solids are washed. Preferably, every approximately 5 gallons of gel, corresponding to about 5 pounds of magnesium hydroxide dry-weight, are washed with approximately 50 gallons of water. The washed magnesium hydroxide is cycled back through the centrifuge and the recovered gel is washed again. Preferably, four washes are conducted to yield magnesium hydroxide of approximately 99+ % purity. For example, the first wash is 10:1 (e.g., 5 gallons gel to 50 gallons water), the second wash is 100:1, the third wash is 1,000:1, and the fourth wash is 10,000:1 in the specified dilution ratios. Depending upon the desired purity of the resulting metal hydroxide nanoplatelets, fewer (e.g., only one, two or three washes) steps can be conducted, or additional (e.g., five or more washes, or other separation processes) steps can be conducted.

The washed magnesium hydroxide nanoplatelets can be employed in subsequent steps in gel form (the product from the centrifuge), or can be subject to drying. In certain embodiments, the magnesium hydroxide can be dried using spray drying equipment using a rotary atomizer or other nozzle configuration. Nozzle inlet temperatures of 280° C. and outlet temperatures of 120° C. for spray drying magnesium hydroxide can be employed; however, any suitable temperature or method for removing liquid from the magnesium hydroxide can be employed.

When the magnesium hydroxide particles in gel form are subjected to drying in a dryer, a particle form referred to as the “Desert Rose configuration” can be obtained. Under the torque of compounding, the lightly-bound together petals of the Desert Rose disassemble to separate nanoplatelets. Alternatively, magnesium hydroxide in the gel state or slurry form can be employed.

Magnesium Hydroxide nanoplatelets in slurry form can be prepared by the method described above. Particularly, the center compartment of the electrolyzer can be filled with RO water containing NaCl at a concentration of 75,000 ppm. The cathode and anode compartments can be subsequently filled with RO water containing NaCl at a concentration of 75,000 ppm. The pressure in the center compartment can be maintained at a higher level than that of the anode and cathode compartments to keep the selective ion membrane in place. The current in the machine can be brought up to 7 volts at 0.75 Amps/Square Inch. The temperature of the contents within the three compartments can be maintained at about 110° F. (about 43° C.) at a pH of about 11. Feedstock can be formulated with a final concentration of Cl⁻ ions at 30,000 ppm and Mg²⁺ ions at 1500 ppm. The feedstock can be fed through the center compartment at a rate of one gallon per minute, resulting in a residence time of 10 minutes. Material can be collected in the catch basin and centrifuged. Slurry collected after centrifugation can be tested to determine the characteristics of the particles within the slurry. The chemical composition of the slurry can be determined by energy dispersive x-ray spectrometric analysis of a dried sample in a JEOL JSM 6500 field emission scanning electron microscope using a Noran Vantage energy dispersive x-ray spectrometer.

To determine particle size and morphology, particles in the slurry can be dispersed in isopropanol, ultrasonicated, and transferred to the analytical substrate in an atomizing spray. Dispersed samples can be prepared on a thin carbon film supported by a standard copper TEM grid. For Field Emission Scanning Electron Microscopy (FESEM) examination, the TEM grid bearing dispersed particles can be placed in a JEOL scanning transmission electron microscopy (STEM) sample holder and the sample holder placed in the FESEM.

STEM images of several fields of view can be obtained using a Noran Vantage digital imaging system, and the diameters of individual particles can be sized using the image processing and particle sizing functions of ImageJ, an image measurement software package distributed and maintained by the National Institutes of Health.

Field Emission Scanning Electron Microscopy (FESEM) can show particles in the form of thin platelets in a narrow size distribution (approximately 50 to 100 nm in the longest dimension). Particle dispersions can be prepared in a similar manner for TEM analysis. Clusters of platelets can be analyzed, with some platelets oriented perpendicular to the viewing axis, allowing measurement of platelet diameter as well as platelet thickness. Transmission Electron Microscopy (TEM) can show particles in the form of thin platelets in a narrow size distribution (e.g., approximately 85% of the particles within 30 to 100 nm in the longest dimension). The TEM images can also show particles with a narrow distribution of thicknesses (e.g., within 1 nm to 5 nm in thickness). Additionally, the TEM images can show particles with a narrow distribution of equivalent spherical diameters (ESDs) (e.g., with approximately 85% of the particles with ESDs of about 15 nm to about 35 nm). Similar particle dispersions as can be prepared for FESEM and TEM can be prepared on mica substrates for atomic force microscopy (AFM) determination of particle diameters, thicknesses, and aspect ratios. Atomic Force Microscopy (AFM) can show particles in the form of thin platelets in a narrow size distribution (with approximately 85% of the particles within 30 to 110 nm in the longest dimension). The AFM can also show particles with a narrow distribution of thicknesses (e.g., 92% of particles were within 1 nm to 5 nm in thickness). Additionally, the AFM can also show particles with Equivalent Spherical Diameters (ESD) within a narrow size distribution (e.g., 93% of particles had an ESD within 15 nm to 40 nm). The average BET surface area can be determined.

The slurry can be tested using scanning electron microscope imaging and analysis by energy dispersive x-ray spectrometry. A portion of the slurry can be diluted in isopropanol and a drop mount prepared on a polished carbon planchet. The prepared sample can be mounted in a JEOL JSM 6500F field emission scanning electron microscope equipped with a Noran Vantage energy dispersive x-ray analysis system. Particle size, morphology, and/or composition can be determined.

In an alternative embodiment, a different electrolyzer configuration is employed to generate the magnesium hydroxide. The electrolyzer includes an anode compartment and a cathode compartment as described above, but with a single ion selective membrane separating the two compartments. NaCl is split in the anode chamber to yield chlorine gas and sodium ion, which passes through the membrane. Water is split in the cathode chamber to yield hydrogen gas and hydroxyl ion. A spacer in the cathode compartment separates the ion selective membrane from the cathode, creating a reaction area. Sodium chloride and metal chloride are added to the cathode chamber. Magnesium ions react with hydroxyl ions in the cathode compartment to yield solid magnesium hydroxide in the cathode chamber leaving a free chloride which combines with sodium from the anode compartment to yield sodium chloride. As in the previously described method, magnesium hydroxide platelet size is determined by adjusting selected variables, as described above. The residence time of feedstock flow through the cathode compartment affects size (faster flow rates result in smaller platelet size), and the quality of flux line by the energy passing between the opposing compartments and temperature affect the speed of reaction. As in the previous method, by adjusting these parameters, magnesium hydroxide platelets of uniform size can be produced. Tight size distributions can be obtained for platelets having an average platelet size of 3.5 microns in the X/Y plane and 100 nm in the Z plane down to particles having an average particle size of 30 nanometers in the X/Y plane and 2.5 nm in the Z plane. Generally, the faster the platelets are harvested, the smaller the resulting platelets. The methods of preferred embodiments can be employed to prepare nanoplatelets over a range of sizes, each having a narrow size distribution. Magnesium hydroxide nanoplatelets having an average platelet diameter of from about 30 nm or less to about 1000, 1500, 2000, 2500, 3300, or 3500 nm or more can be prepared, for example, from about 40, 50, 60, 70, 80, 90, 100, 110, 120, 130, 140, 150, 160, 170, 180, or 190 nm to about 200, 250, 300, 350, 400, 450, 500, 600, 700, 800, or 900 nm. Particularly preferred are nanoplatelets having an average platelet diameter of about 40, 50, or 60 nm to about 70, 80, 90, 100, 110, or 120 nm.

### Preparation of Metal Hydroxide Monolayer

A method of producing monolayer metal hydroxides of superior properties is desirable. The materials and methods disclosed herein can be employed to prepare such metal hydroxides to form a monolayer nanoplatelet.

Nanoplatelets of metal hydroxide are mixed into a water column or other suitable fluid (e.g., a liquid such as acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol dimethyl ether), 1,2-dimethoxy-ethane (glyme, DME), dimethyl-formamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous triamide (HMPT), hexane, methanol, methyl t-butyl ether (MTBE), methylene chloride, N-methyl-2-pyrrolidinone (NMP), nitromethane, pentane, Petroleum ether (ligroine), 1-propanol, 2-propanol, pyridine, tetrahydrofuran (THF), toluene, triethyl amine, water, o-xylene, m-xylene, p-xylene, or the like), as a precursor from which the core of a monolayer nanoplatelet is formed. Metal salts or other metal ion sources are present (dissolved) in the water column or other suitable fluid to supply the ions to self assemble, by ion exchange, a monolayer shell. By this process, a metal hydroxide monolayer nanoplatelet is created.

The monolayer shell is formed by ion exchange from the metal hydroxide core with a less reactive metal ion from the water column or other suitable fluid, thereby reducing that shell species concentration in the fluid column and increasing core ion content of the fluid column. Metal hydroxide monolayer nanoplatelets with the core comprised of a more reactive metal hydroxide, and a less reactive metal hydroxide shell, wherein the shell metal is different from the core metal. The metal hydroxide nanoplatelet typically has a diameter of from about 30 nm to about 3500 nm, a thickness of from about 1 nm to about 400 nm, and an aspect ratio of from 15 to 75, and comprises individual crystallites; however, different dimensions are contemplated. It is generally preferred not to have nanoplatelets having a diameter of less than about 30 nm, in that they tend not to maintain structural integrity in terms of their dimensions (e.g., they decompose or degrade upon exposure to water or other ambient conditions as disclosed herein).

In one embodiment, the magnesium hydroxide nanoplatelets are subjected to ion exchange to yield copper hydroxide nanoparticles. The magnesium hydroxide nanoplatelets are exposed to copper ion. The copper ion replaces the magnesium ion in the nanoparticles, in part. In certain embodiments, a copper salt (e.g., copper chloride) in solid form or in a form of a copper ion solution is added to an aqueous slurry of magnesium hydroxide nanoplatelets. In other embodiments, magnesium hydroxide nanoparticles in a dry form are added to a solution of copper salt. The amount of copper ion can be selected based on the amount of magnesium ion to be exchanged. For example, less than stoichiometric amounts of copper ion can be provided if partial exchange is desired. Stoichiometric amounts of copper ion can be provided for efficient exchange of the magnesium ion present, or an excess of copper ion can be provided. In certain embodiments, a saturated solution of copper ion is provided, however, in certain embodiments a solution that is not saturated can be provided. Any suitable method for mixing or combining can be employed. The ion exchange can advantageously be conducted at ambient temperatures (e.g., approximately 20° C.), however, in certain other embodiments a liquid mixture of a higher or lower temperature can be employed. The temperature and exposure time can be adjusted such that different degrees of conversion of ion exchange can be achieved. For example, a monolayer of copper hydroxide surrounding a core of magnesium hydroxide can be obtained. In other embodiments, full replacement of the magnesium can be obtained yielding a copper hydroxide nanoparticle containing negligible magnesium. In other embodiments, the amount of replacement can vary from full replacement to partial replacement (a nanometer of surface replacement or less). A slurry of magnesium hydroxide particles before treatment is milky white and after treatment with a copper chloride solution is a cloudy light blue.

Methods and methodologies as described herein can be used to produce monolayer nanoplatelets. The base materials used to make the process feedstock include, for example, magnesium hydroxide nanoplatelets for the core and copper chloride or another soluble copper salt for formation of a shell. Suitable shell materials include commercially available bulk forms of copper chloride. While copper chloride is particularly preferred, other sources of soluble copper ion can also be employed, for example, other copper halides such as copper bromide, copper iodide, copper nitrate, and copper sulfide. Any suitable nanoparticulate form of magnesium hydroxide can be used as a starting core material; however, magnesium hydroxide as prepared according to the method described herein or any other method can advantageously be employed.

As noted earlier, the term “metal hydroxide” is employed to refer to a metal hydroxide, a metal oxide, or mixtures thereof. Metal oxides and hydroxides more reactive to form the core, and less reactive than the shell ions. The core Metal oxides and hydroxides is not limited to, scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, ununbium lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, and lawrencium, mixed oxides and hydroxides of the foregoing metals, mixed metal oxides and/or hydroxides, and other combinations thereof.

The shell is formed from ions of less reactive metal ions than the core of metal oxides and/or hydroxides to form the monolayer shell of metal oxide and/or hydroxides by ion exchange. The shell metal can be an ion of, e.g., scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, ununbium lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, or lawrencium, or any combination thereof.

### Pharmaceutical Uses

Magnesium hydroxide nanoparticles as described herein are useful pharmaceutical agents for the treatment and/or prophylaxis of certain conditions. These conditions include infection by microorganisms (including but not limited to viruses, bacteria, and fungus), promoting healing of wounds, treatment of injuries to the skin (thermal burns, radiation burns, chemical burns, sunburns, contact dermatitis, shingles, eczema, abrasions), providing pain relief to damaged or irritated skin, and the treatment of certain types of cancer (including but not limited to skin cancer). The magnesium hydroxide nanoparticles are useful as ethical drugs (also referred to as prescription drugs), as well as over-the-counter formulations.

The magnesium hydroxide nanoparticles are efficacious in treating infections of the respiratory system and the skin, but are also useful in treating other affected parts of the body, including systemic infections.

A lung infection can be caused by a virus, bacteria, or a fungus. One of the most common types of lung infections is pneumonia. Pneumonia, which affects the smaller air sacs of the lungs, is most often caused by contagious bacteria, but can also be caused by a virus. A person becomes infected by breathing in the bacteria or virus after a nearby infected person sneezes or coughs. When the large bronchial tubes that carry air to and from the lungs become infected, it is referred to as bronchitis. Bronchitis is more likely to be caused by a virus than by bacteria. Viruses can also attack the lungs or the air passages that lead to the lungs. This is called bronchiolitis. Viral bronchiolitis most commonly occurs in infants.

Lung infections like pneumonia are usually mild, but they can be serious, especially for people with weakened immune systems or chronic conditions, such as chronic obstructive pulmonary disease (COPD), or comorbidities such as lung based, small cell carcinoma or mesothelioma.

The most common microorganisms responsible for bronchitis include viruses such as the influenza virus or respiratory syncytial virus (RSV), and coronaviruses, bacteria such as Mycoplasma pneumoniae, Chlamydia pneumoniae, Bordetella pertussis, Streptococcus pneumonia, Haemophilus influenzae, and Mycoplasma pneumoniae. Lung infections can be caused by fungi such as Pneumocystis jirovecii, Aspergillus, or Histoplasma capsulatum. A fungal lung infection is more common in people who are immunosuppressed, either from certain types of cancer or HIV or from taking immunosuppressive medications.

Examples include coronaviruses, such as those that can cause pulmonary effects. These include the common cold, SARS-associated coronavirus (SARS-CoV, SARS-CoV-2), MERS-associated corona virus (MERS-CoV), COVID-19, influenza viruses such as Influenza A (H1N1) virus, Zika virus (a member of the virus family Flaviviridae), and Marburg virus.

The magnesium hydroxide nanoparticles are also useful in preventing or treating other respiratory illnesses and diseases, or alleviating certain symptoms associated with respiratory illnesses and diseases (e.g., through antimicrobial activity or by attacking abnormal cells or other mechanisms of action). These other respiratory illnesses and diseases include but not are not limited to asthma, chronic obstructive pulmonary disease (COPD), chronic and acute bronchitis, emphysema, lung cancer or cancer of other respiratory structures, cystic fibrosis/bronchiectasis, pneumonia, pulmonary edema, acute respiratory distress syndrome (ARDS), pneumoconiosis, interstitial lung disease, mesothelioma, pneumothorax, and pleural effusion.

Skin infections include bacterial infections (e.g., cellulitis, impetigo, boils, leprosy), viral infections (e.g., shingles (herpes zoster), chickenpox, Molluscum contagiosum, warts, measles, hand, foot, and mouth disease), fungal infections (e.g., athlete's foot, yeast infection, ringworm, nail fungus, oral thrush, diaper rash), parasitic skin infections (e.g., lice, bedbugs, scabies, cutaneous larva migrans). A patient having a skin infection may be asymptomatic, or may exhibit mild to severe symptoms (e.g., pus, blisters, skin sloughing or breakdown, necrotic-appearing or discolored skin, pain, irritation, itching).

Skin can suffer injury from a variety of causes in addition to infection. The compositions described herein are used to promote healing and to treat symptoms associated with skin injury, including physical damage, pain, pruritus, inflammation, and irritation due to a variety of factors and conditions. Non-limiting examples include allergies, insect bites (e.g., hymenoptera, fleas, bed bugs, spiders, ants, ticks, etc.), stinging animals (e.g., jellyfish, scorpions, caterpillars, etc.) delayed type hypersensitivity, hives, exposure to venom, poison ivy, atopic dermatitis, eczema, acne, psoriasis, rosacea, ichthyosis vulgaris, dermatomyositis, thermal burns, herpes, ionizing radiation, exposure to chemicals, trauma, surgery, nerve compression, back pain, amputation, trauma, oral or throat ulcers, post herpetic neuralgia, multiple sclerosis, Parkinson's disease, lupus, diabetes, pressure sores, skin plaques, ulcers, scale, dermatoses, hives, blisters, warts, shingles, boils, diabetic neuropathy, rheumatoid arthritis, psoriatic arthritis, ankylosing spondylitis, idiopathic arthritis, cold sores, and drug use.

The magnesium hydroxide nanoparticles are designed to be topically applied for the treatment of skin conditions and conditions of the mucous membranes. Topical application in its broadest sense means application to epithelial surfaces such as skin or mucous membranes, including eyes, mouth, throat, esophagus, gastrointestinal tract, respiratory tract, and genitourinary tract.

The magnesium hydroxide nanoparticles can be applied post incident or upon development of the condition, e.g., application after exposure to poison ivy, after receiving an insect bite, after development of a burn such as sunburn, after lesions or blisters develop, etc. Such application can alleviate symptoms or promote a faster healing time. Alternatively, the magnesium hydroxide nanoparticles can be regularly applied in the initial onset of symptoms or during the early stages of a condition to reduce or minimize the symptoms or skin damage associated with a condition, e.g., to a cold sore area when the skin begins to itch, early stage psoriatic plaque, immediately after sunburn, etc. The magnesium hydroxide nanoparticles can be applied in a preventative manner to reduce of minimize the symptoms or skin damage that normally occurs with a given condition, apply to a shingles rash area before the development of the rash, or to skin prior to a radiation treatment for cancer, etc. The magnesium hydroxide nanoparticles can be applied on a regular basis as part of a normal daily skin care routine. The compositions can be used immediately after a traumatic event. Non-limiting examples of traumatic events include puncture wounds, cuts, abrasions, surgical incisions, amputation, burns (1^{st}, 2^{nd}, 3^{rd}, or 4^{th} degree burns, e.g., deep tissue burns caused by radiation or thermal energy), compound or open bone fractures, and shingles.

Certain cancers may also be treatable by the compositions. There are three major types of skin cancers: basal cell carcinoma (BCC), squamous cell carcinoma (SCC), and melanoma. The first two skin cancers are grouped together as non-melanoma skin cancers. Other unusual types of skin cancer include Merkel cell tumors and dermatofibrosarcoma protruberans. Precancerous lesions and dysplasias include actinic keratosis and dysplastic nevi. The compositions can be applied to the abnormal cells to treat the cancer to dysplasia. While not wishing to be bound by theory, it is believed that the cancerous or precancerous cells are susceptible to lysing by penetration by the nanoparticles, and that noncancerous cells, which may have a thicker outer layer, are resistant to such penetration. Other types of cancer cells (carcinomas, sarcomas, leukemias, lymphomas, myelomas) may be susceptible to treatment by contact with the compositions, including but not limited to lung cancer (small cell and large cell), breast cancer, prostate cancer, colorectal cancer, bladder cancer, or to any other cancer cell susceptible to penetration by the nanoparticles. The magnesium hydroxide nanoparticles can be applied directly to the cells (e.g., by injection, implantation, systemic delivery, or topical delivery), or can be applied to a tumor bed after removal of a tumor to prevent or inhibit recurrence of the cancer.

Pharmaceutical compositions including magnesium hydroxide nanoparticles are provided. The magnesium hydroxide nanoparticles can be as described in U.S. Pat. No. 7,892,447, incorporated by reference herein in its entirety. In certain embodiments, the magnesium hydroxide nanoparticles are nanoplatelets having a narrow distribution of thicknesses in the form of thin platelets in a narrow size distribution (with approximately 85% of the particles within 30 to 100 nm in the longest dimension). In certain embodiments, the magnesium hydroxide nanoparticles can have a narrow distribution of thicknesses (within 1 nm to 5 nm in thickness). In certain embodiments, the magnesium hydroxide nanoparticles have an average aspect ratio of from about 15 to about 70, an average platelet diameter of from about 30 nm to about 3500 nm and an average thickness of from about 2.5 nm to 100 nm, and optionally an average BET specific surface area of from about 100 m²/g to about 150 m²/g. The pharmaceutical compositions can optionally include at least one excipient, depending upon the route of administration.

In certain embodiments, the pharmaceutical compositions can optionally include zinc (known to possess anti-viral activity) either incorporated into or onto the nanoparticles (e.g., as a dopant, as a monolayer or thicker coating, in ionic form, in a form of zinc oxide), or as a separate component of the composition. When zinc is incorporated into the nanoparticle, the methods as described here for preparing copper hydroxide monolayer nanoparticles can be readily adapted to produce zinc hydroxide monolayer nanoparticles, e.g., by ion exchange with a zinc salt.

It is also generally preferred to administer the compositions through inhalation (e.g., as a vapor, a mist, or an aerosol), other routes of administration are also contemplated. Delivery devices include inhalers, humidifiers, and the like. The compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions disclosed herein may be manufactured by a process that is itself known, e.g., by means of conventional mixing, dissolving, emulsifying, or extracting processes. Many of the compounds used in the pharmaceutical compositions may be provided as salts with pharmaceutically acceptable counterions.

Multiple techniques of administering pharmaceutical compositions exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein are any methods suitable for administering the composition to a portion of the respiratory system, or skin, or tumor, or other target area (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the magnesium hydroxide nanoparticles may be combined as an active ingredient in intimate admixture with a pharmaceutical carrier according to conventional pharmaceutical compounding techniques. The carrier can take a wide variety of forms depending on the form of preparation desired for administration. Thus, the compositions provided herein can be presented as discrete units suitable for pulmonary administration such as vials containing a predetermined amount of the active ingredient(s), optionally with accessories such as inhalers or nebulizers. Further, the magnesium hydroxide nanoparticles can be presented as an aqueous or nonaqueous suspension, as an emulsion, or on or in a carrier as employed for providing a pharmaceutical composition to the lungs, skin, tumor, or other target area. In addition to the common dosage forms set out above, the magnesium hydroxide nanoparticles can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both.

The magnesium hydroxide nanoparticles can be provided via a humidifier (e.g., an ultrasonic, mist, or vaporizer humidifier) as are commonly available over-the-counter. Other devices for administering include inhalers and nebulizers. The most common type of inhaler is the pressurized metered-dose inhaler (MDI) which is made up of 3 standard components-a metal canister, plastic actuator, and a metering valve. In MDIs, medication is typically stored in solution in a pressurized canister that contains a propellant, although it may also be a suspension. The MDI canister is attached to a plastic, hand-operated actuator. On activation, the metered-dose inhaler releases a fixed dose of medication in aerosol form. The correct procedure for using an MDI is to first fully exhale, place the mouth-piece of the device into the mouth, and having just started to inhale at a moderate rate, depress the canister to release the medicine. The aerosolized medication is drawn into the lungs by continuing to inhale deeply before holding the breath for 10 seconds to allow the aerosol to settle onto the walls of the bronchittus and other airways of the lung. Dry powder inhalers (DPI) can also advantageously be employed. DPI release a metered or device-measured dose of powdered medication that is inhaled through a DPI device. Nebulizers supply the medication as an aerosol created from an aqueous formulation. Nasal inhalers deliver drugs to the upper respiratory tract. Propellants for inhalers include hydrofluoroalkane (HFA).

The pharmaceutical compositions contain the magnesium hydroxide nanoparticles optionally in combination with zinc in an amount effective for the desired therapeutic effect. In some embodiments, the magnesium hydroxide nanoparticles are provided in a carrier (e.g., water) at a concentration of 0.001% to 10% by weight. Depending upon the mode of delivery, higher or lower concentrations may be employed. The magnesium hydroxide nanoparticles can be provided in a unit dosage form and comprise from about 0.01 mg or less to about 5000 mg or more of magnesium hydroxide nanoparticles per unit dosage form. Such dosage forms may be provided in a ready to use form, or can be reconstituted in a suitable carrier fluid for delivery via aerosol, mist, vapor, or the like for inhalation administration. Other formulations include topical formulations comprising the magnesium hydroxide nanoparticles in a carrier (e.g., creams, gels, ointments, sprays). In certain embodiments, the magnesium hydroxide nanoparticles can be administered as a powder, either as a pure form (100% by weight magnesium hydroxide nanoparticles) or with a suitable diluent or carrier.

The magnesium hydroxide nanoparticles can be prepared as suspensions of the magnesium hydroxide nanoparticles in water or oil or other liquid carrier. A suitable surfactant can be included such as, for example, hydroxypropylcellulose. Dispersions can also be prepared in glycerol, liquid polyethylene glycols, and mixtures thereof in oils.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Compositions containing magnesium hydroxide nanoparticles provided herein can also be prepared in powder or liquid concentrate form for dilution.

The magnesium hydroxide nanoparticles and other active ingredient(s) (e.g., zinc) may be present in a single formulation or in multiple formulations provided together, or may be unformulated. In some embodiments, the magnesium hydroxide nanoparticles can be administered with one or more additional agents together in a single composition. For example, the magnesium hydroxide nanoparticles can be administered in one composition, and the zinc can be administered in a second composition. In a further embodiment, the magnesium hydroxide nanoparticles and zinc are co-packaged in a kit. For example, a drug manufacturer, a drug reseller, a physician, a compounding shop, or a pharmacist can provide a kit comprising magnesium hydroxide nanoparticles optionally with zinc or other active ingredients for delivery to a patient.

In some embodiments, magnesium hydroxide nanoparticles may be provided in a kit comprising the components necessary to prepare the magnesium hydroxide nanoparticles for delivery in a therapeutic solution. In some embodiments, the kit may comprise the magnesium hydroxide nanoparticles in a solid (dry) form and a carrier, e.g., an aqueous solution, e.g., saline solution, or a propellant for pulmonary delivery. The kit may be configured to optimize the storage conditions of the magnesium hydroxide nanoparticles, for short or long-term storage. In some embodiments, the kit may be configured to store the magnesium hydroxide nanoparticles for up to at least 1 week, 2 weeks, 3 weeks, 4 weeks, 1 month, 2 months, 3 months, 4 months, 5 months, 6 months, 1 year, 2 years, or 3 years. The kit may comprise one or more aliquots of each component in pre-measured amounts or volumes. Each component may be provided in a sealed vial, tube, or other container as is known in the art. The containers may each comprise plastic and/or glass. The containers may be configured (for example, tinted or covered) to protect the components from light and/or other radiation. In some embodiments, the kit may be configured for shipping. For example, the components may be contained in a box or other container including desiccants and/or may be configured for temperature control. In some embodiments, the magnesium hydroxide nanoparticles and/or other components may be supplied in a container that has been purged of air (e.g., oxygen). The magnesium hydroxide nanoparticles may be stored under vacuum or may be purged with an inert gas, such as nitrogen or argon. In some embodiments, the magnesium hydroxide nanoparticles may be mixed with a stabilizer, in addition to or alternatively to purging the air. In some embodiments, the magnesium hydroxide nanoparticles may be provided already suspended in a carrier liquid to a predetermined concentration. In some embodiments, the volume of water, saline or other liquid carrier provided may be configured to prepare the magnesium hydroxide nanoparticles at a desired therapeutic concentration. In some embodiments, the volume of liquid may be configured to prepare the magnesium hydroxide nanoparticles at a maximal therapeutic concentration, such that a user may dilute the magnesium hydroxide nanoparticles with additional liquid to the desired therapeutic concentration. In some embodiments, the total volume of liquid may be configured to prepare the magnesium hydroxide nanoparticles at a concentration below the desired concentration and the user may use only a portion of the volume of the liquid to prepare the magnesium hydroxide nanoparticles to the desired concentration. The container of suspended magnesium hydroxide nanoparticles may have volume indicators for facilitating measurement of the liquid. In some embodiments, the liquid may be provided in a plurality of aliquots having the same and/or different volumes, which may allow the user to select an aliquot of a desired volume to prepare the magnesium hydroxide nanoparticles at a desired concentration and/or combine various volumes to prepare the magnesium hydroxide nanoparticles at a desired concentration. In some embodiments, the kit may comprise one or more additional components. For example, the kit may comprise a zinc-containing component for mixing with the therapeutic magnesium hydroxide nanoparticles suspension.

In certain embodiments, the magnesium hydroxide nanoparticles can be administered in an injectable form, e.g., via a delivery catheter or syringe directly to a tumor treatment site; however, other routes of administration are also contemplated. Contemplated methods of administration include but are not limited to orally, subcutaneously, intravenously, intranasally, topically, transdermally, intraperitoneally, intramuscularly, intrapulmonarilly, vaginally, rectally, or intraocularly. The magnesium hydroxide nanoparticles can be formulated into liquid preparations for, e.g., oral administration, or solid forms, e.g., tablets for ingestion or implants for seeding a tumor. Other suitable forms include suspensions, syrups, elixirs, and the like. Unit dosage forms for oral administration include tablets and capsules. Unit dosage forms configured for administration once a day can be employed; however, in certain embodiments it can be desirable to configure the unit dosage form for administration twice a day, or more.

Depending upon the particular route of administration of the magnesium hydroxide nanoparticles desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the nanoparticles. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, for example, in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions. Tonicity adjustors may be added as needed or convenient. They include, but are not limited to, salts, particularly sodium chloride, potassium chloride, mannitol and glycerin, or any other suitable ophthalmically acceptable tonicity adjustor. Various buffers and means for adjusting pH may be used so long as the resulting preparation is pharmaceutically acceptable. For many compositions, the pH will be between 4 and 9. Accordingly, buffers include acetate buffers, citrate buffers, phosphate buffers and borate buffers. Acids or bases may be used to adjust the pH of these formulations as needed. Acceptable antioxidants include, but are not limited to, sodium metabisulfite, sodium thiosulfate, acetylcysteine, butylated hydroxyanisole and butylated hydroxytoluene. A useful chelating agent is edetate disodium, although other chelating agents may also be used in place or in conjunction with it. Topical formulations may generally be comprised of the magnesium hydroxide nanoparticles, a pharmaceutical carrier, emulsifier, penetration enhancer, preservative system, and emollient.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (for example, from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The magnesium hydroxide nanoparticles formulation can be in a solid form, or in a liquid form, such as a viscous liquid form. Viscosity of the formulation can be maintained at the selected level using a pharmaceutically acceptable thickening agent. Methylcellulose is readily available and is easy to work with. Other suitable thickening agents include, for example, xanthan gum, carboxymethyl cellulose, hydroxypropyl cellulose, carbomer, and the like. The concentration of the thickener will depend upon the thickening agent selected. An amount is typically used that will achieve the selected viscosity. Viscous compositions are normally prepared from solutions by the addition of such thickening agents, and are suitable for topical use.

The PGG can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

For oral administration, the magnesium hydroxide nanoparticles can be provided as a tablet, aqueous or oil suspension, dispersible powder or granule, emulsion, hard or soft capsule, syrup or elixir. Compositions intended for oral use can be prepared according to any method known in the art for the manufacture of pharmaceutical compositions and can include one or more of the following agents: sweeteners, flavoring agents, coloring agents and preservatives. Aqueous suspensions can contain the active ingredient in admixture with excipients suitable for the manufacture of aqueous suspensions.

Magnesium hydroxide nanoparticles formulations for oral use can be solid forms as tablets, capsules, granules and bulk powders, or can be provided as hard gelatin capsules, wherein the active ingredient(s) are mixed with an inert solid diluent, such as calcium carbonate, calcium phosphate, or kaolin, or as soft gelatin capsules. In soft capsules, the active compounds can be dissolved or suspended in suitable liquids, such as water or an oil medium, such as peanut oil, olive oil, fatty oils, liquid paraffin, or liquid polyethylene glycols. Stabilizers and microspheres formulated for oral administration can also be used. Capsules can include push-fit capsules made of gelatin, as well as soft, sealed capsules made of gelatin and a plasticizer, such as glycerol or sorbitol. The push-fit capsules can contain the active ingredient in admixture with fillers such as lactose, binders such as starches, and/or lubricants such as talc or magnesium stearate and, optionally, stabilizers.

Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents. Tablets can be uncoated or coated by known methods to delay disintegration and absorption in the gastrointestinal tract and thereby provide a sustained action over a longer period of time. For example, a time delay material such as glyceryl monostearate can be used. When administered in solid form, such as tablet form, the solid form typically comprises from about 0.001 wt. % or less to about 50 wt. % or more of active ingredient(s), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets can contain the magnesium hydroxide nanoparticles in admixture with non-toxic pharmaceutically acceptable excipients including inert materials. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art. For example, a tablet can be prepared by compression or molding, optionally, with one or more additional ingredients. Compressed tablets can be prepared by compressing in a suitable machine the active ingredients in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Preferably, each tablet or capsule contains from about 10 mg or less to about 1,000 mg or more of a compound of the magnesium hydroxide nanoparticles, more preferably from about 20, 30, 40, 50, 60, 70, 80, 90, or 100 mg to about 150, 200, 250, 300, 350, 400, 450, 500, 550, 600, 650, 700, 750, 800, or 900 mg. Most preferably, tablets or capsules are provided in a range of dosages to permit divided dosages to be administered. A dosage appropriate to the patient and the number of doses to be administered daily can thus be conveniently selected. In certain embodiments it can be preferred to incorporate the magnesium hydroxide nanoparticles and one or more other therapeutic agents to be administered into a single tablet or other dosage form (e.g., in a combination therapy, e.g., with zinc); however, in other embodiments it can be preferred to provide the magnesium hydroxide nanoparticles and other therapeutic agents in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents can be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya or tragacanth, or alginic acid or salts thereof.

Binders can be used to form a hard tablet. Binders include materials from natural products such as acacia, tragacanth, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, can be included in tablet formulations.

Surfactants can also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations of magnesium hydroxide nanoparticles can be employed wherein the magnesium hydroxide nanoparticles is incorporated into an inert matrix that permits release by either diffusion or leaching mechanisms, e.g., a microencapsulated form. Slowly degenerating matrices can also be incorporated into the formulation. Other delivery systems can include timed release, delayed release, or sustained release delivery systems. These forms are suitable for use in seeding a solid tumor.

Coatings can be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments can be added for identification or to characterize different combinations of active compound doses. Coatings can include pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

When administered orally in liquid form, a liquid carrier such as water, petroleum, oils of animal or plant origin such as peanut oil, mineral oil, soybean oil, or sesame oil, or synthetic oils can be added to the active ingredient(s). Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above. Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents, or, in the case of topical formulations, fragrances. Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

When magnesium hydroxide nanoparticles is administered by intravenous, parenteral, or other injection, it is preferably in the form of a pyrogen-free, parenterally acceptable aqueous solution, alcoholic solution (e.g., ethanolic solution), or oleaginous suspension. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred pharmaceutical composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed including synthetic mono or diglycerides. In addition, fatty acids such as oleic acid can likewise be used in the formation of injectable preparations. The pharmaceutical compositions can also contain stabilizers, preservatives, buffers, antioxidants, or other additives known to those of skill in the art.

The duration of an intravenous injection or other injection can be adjusted depending upon various factors, and can comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous or other injection administration.

In some embodiments, the magnesium hydroxide nanoparticles are formulated for administration by inhalation. Various forms suitable for administration by inhalation include, but are not limited to, aerosols, mists or powders. In some embodiments, the active agent or agents are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide, and/or other suitable gases). In some embodiments, the dosage unit of a pressurized aerosol is determined by providing a valve to deliver a metered amount. In some embodiments, capsules and cartridges of, such as, by way of example only, gelatins for use in an inhaler or insufflator are formulated containing a powder mix of magnesium hydroxide nanoparticles optionally with a zinc containing-component as disclosed elsewhere herein and a suitable powder base such as lactose or starch.

The magnesium hydroxide nanoparticles compositions of the preferred embodiments can additionally employ adjunct components conventionally found in pharmaceutical compositions in their art-established fashion and at their art-established levels. Thus, for example, the compositions can contain additional compatible pharmaceutically active materials for therapy (such as antimicrobials, local anesthetics, anti-inflammatory agents, and the like), or can contain materials useful in physically formulating various dosage forms of the preferred embodiments, such as excipients, dyes, thickening agents, stabilizers, preservatives or antioxidants.

Some embodiments described herein relate to a composition, which can include a therapeutically effective amount of magnesium hydroxide nanoparticles optionally with zinc. The pharmaceutical composition can include magnesium hydroxide nanoparticles in, for example, >0.001%, ≥0.01%, ≥1%, ≥2%, ≥3%, ≥4%, ≥5%, ≥6%, ≥7%, ≥8%, ≥9%, ≥10%, ≥20%, or more by weight of the composition. In some embodiments, the pharmaceutical composition can include the zinc in, for example, >0.001%, ≥0.01%, ≥1%, ≥2%, ≥3%, ≥4%, ≥5%, ≥6%, ≥7%, ≥8%, ≥9%, ≥10%, ≥20%, or more by weight of the composition.

Provided herein are compositions and methods for treating pulmonary infections, e.g., viral pulmonary infections such as COVID-19, respiratory conditions, skin conditions, and certain cancers. These conditions are treated by administration of magnesium hydroxide nanoparticles optionally with zinc, optionally in a suitable carrier.

As will be readily apparent to one skilled in the art, the useful in vivo dosage to be administered and the particular mode of administration will vary depending upon the age, weight, the severity of the condition, and mammalian species treated, the particular forms of the components employed, and the specific use for which these components are employed. The determination of effective dosage levels, that is the dosage levels necessary to achieve the desired result, can be accomplished by one skilled in the art using routine methods, for example, in vivo studies. Reference may be made to, for example, “Estimating the Maximum Safe Starting Dose in Initial Clinical Trials for Therapeutics in Adult Healthy Volunteers,” U.S. Food and Drug Administration, July 2005.

In some embodiments, a method provided herein may comprise administering a therapeutically effective amount of magnesium hydroxide nanoparticles as provided herein.

The dosage may vary broadly, depending upon the desired effects and the therapeutic indication. Alternatively, dosages may be based and calculated upon the surface area or weight of the patient, as understood by those of skill in the art. The exact dosage will be determined on a case-by-case basis, or, in some cases, will be left to the informed discretion of the subject. The daily dosage regimen for an adult human patient may be, for example, a dose of the magnesium hydroxide nanoparticles of from about 0.01 mg to about 10000 mg, from about 1 mg to about 5000 mg, from about 5 mg to about 2000 mg, from about 10 mg to about 1000 mg, or from about 50 mg to about 500 mg. Zinc, if present, may be administered in a dose of about 0.01 mg, about 0.1 mg, about 1 mg, about 5 mg, about 10 mg, about 20 mg, about 50 mg, about 100 mg, about 200 mg, about 300 mg, about 400 mg, about 500 mg, about 600 mg, about 800 mg, about 900 mg, about 1000 mg, about 2000 mg, about 5000 mg, or more. The dosage may be adjusted according to the body mass of the subject, for example, the dosage may be about 0.001 mg/kg, about 0.01 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, or higher of magnesium hydroxide nanoparticles, optionally with zinc in an amount of about 0.001 mg/kg, about 0.01 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, or higher.

The dosage may be administered as a single dosage one or a series of two or more dosages given in the course of one or more days, as is appropriate for the individual subject. In some embodiments, e.g., for pulmonary or respiratory treatment, e.g., by aerosol, mist, or nebulizer, the composition can be administered until symptoms subside, or for a period of continuous therapy, for example for about one day, two days, three days or more, or a week or more (e.g., one week, two weeks, three weeks, or more). In some embodiments, the combination can be administered or inhaled one time per day, two times per day, three times per day, or more, or continuously. For skin conditions, the topical composition can be administered once, twice, three times, or more per day, for from one day to one or more weeks. For tumors, the composition can be injected, implanted, infused, or administered systemically until a desired endpoint is reached (e.g., tumor shrinkage, reduction in amount of cancer cells, reduction in a marker of cancer, or any other clinically accepted endpoint).

As will be understood by those of skill in the art, in certain situations it may be necessary to administer the magnesium hydroxide nanoparticles in amounts that exceed the above-stated, preferred dosage range in order to effectively treat a subject.

Unit dosage forms can also be provided, e.g., individual vials with a premeasured amount of the magnesium hydroxide nanoparticles, configured for administration on a predetermined schedule. Unit dosage forms configured for administration one to three times a day are preferred; however, in certain embodiments it may be desirable to configure the unit dosage form for administration more than three times a day, or less than one time per day, or for continuous administration.

Dosage amounts and intervals may be adjusted to the individual subject to provide levels of the magnesium hydroxide nanoparticles which are sufficient to maintain predetermined parameters, indicators, or marker values, or minimal effective concentration (MEC). Dosages necessary to achieve the desired result will depend on individual characteristics and route of administration. However, assays, for example, HPLC assays or bioassays, may be used to determine concentrations.

### Treatment of Toxic Waste

While copper hydroxide nanoparticles (or copper hydroxide coated magnesium hydroxide nanoparticles) can be advantageously prepared using the ion exchange method described above, the method can also be used to prepare other metal hydroxide nanoparticles. In such cases, magnesium ion is more labile than the other metal with respect to association with hydroxide. Other metals besides copper that can be employed in ion exchange include the rare earth elements and transition metals (including the noble metals). Transition metals include scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, gold, mercury, rutherfordium, dubnium, seaborgium, bohrium, hassium, meitnerium, ununnilium, unununium, and ununbium. Rare earth elements include the lanthanide series (lanthanum, cerium, praseodymium, neodymium, promethium, samarium, europium, gandolinium, terbium, dysoprosium, holmium, erbium, thulium, ytterbium, lutetium) and the actinide series (actinium, thorium, protactinium, uranium, neptumium, plutonium, americium, curium, berkelium, californium, einsteinium, fermium, mendelevium, nobelium, and lawrencium). The resulting nanoparticles may advantageously be employed for various uses as a valuable material in their own right.

Alternatively, magnesium hydroxide nanoparticles can be added to a toxic waste stream or volume to advantageously absorb undesirable (e.g., toxic, radioactive, etc.) metal ions by ion or elemental exchange, sequestering the metal in the metal hydroxide nanoparticles while releasing magnesium ion as a byproduct. The methodology can be used to treat a waste stream such that amounts of radioactive or toxic metals are reduced below safe EPA discharge limits. The methodology is also amenable to use in treatment of large volumes of contaminated water or other waste stream. Magnesium hydroxide nanoparticles are particularly suited for remediation of fly ash and mine tailings, which typically have high levels of undesirable metals. The magnesium hydroxide also has antimicrobial properties, and as such can provide remediation of undesirable microbes in addition to metal sequestration.

Once the ion exchange process has been completed to a desired degree, the metal nanoparticles can be separated and either disposed of or subjected to further processing, e.g., to recover the sequestered metal and convert it to a more valuable form, e.g., metallic form. For example, in an alternative method, magnesium hydroxide nanoparticles can be employed to scavenge valuable ions (e.g., of rare earth metals or noble metals). The resulting metal nanoplatelets can then be subjected to, e.g., electrowinning or thermal reduction to secure the metal in a purified form. Such a process can be employed in mining of raw materials or in other resource extraction processes, e.g., recovery of metals from recycled electronics or other metal sources.

Magnesium hydroxide nanoparticles can be added to a toxic waste stream or volume of a polar fluid (e.g., an aqueous liquid) to advantageously absorb undesirable (e.g., toxic, radioactive, etc.) metal ions by ion or elemental exchange, thereby sequestering the metal in the metal hydroxide monolayer nanoparticles shell while releasing magnesium ion as a byproduct. The methodology can be used to treat a waste stream such that amounts of radioactive or toxic metals are reduced below safe EPA discharge limits. The methodology is also amenable to use in treatment of large volumes of contaminated water or other waste streams. Magnesium hydroxide nanoparticles are particularly suited for remediation of fly ash and mine tailings, which typically have high levels of undesirable metals, including but not limited to scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, ununbium lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, and lawrencium. The metal monolayer nanoparticles also have antimicrobial properties, and as such can provide remediation of undesirable microbes in addition to metal ion sequestration. Once the ion exchange process has been completed to a desired degree, the metal monolayer nanoparticles can be separated and disposed of. A sample of fly ash water (first sample) that was untreated was clear with a straw-colored hue. Treated fly ash water was clear (second and third samples) to slightly cloudy (fourth sample), each with a white sediment on the bottom of the sample container. Table 1 provides analytical results for the untreated fly ash water, which show the untreated fly ash water was above the discharge limits of the EPA Clean Water Act. Table 2 shows analytical results for the treated third sample, which was below the Clean Water Act discharge limit as to contaminant elements tested.

TABLE 1

| | | | Method | |
|---|---|---|---|---|
| | | | Quantification | Analytical |
| Test | Results | Units | Limit | Method |
| Fluoride (w/o | 2.8 | mg/L | 0.125 | EPA-300 |
| distillation) | | | | |
| Nitrate (NO3—N) | 4.38 | mg/L | 0.100 | EPA-300 |
| Total Dissolved Solids | 517 | mg/L | 50.5 | 2540C-2011 |
| Arsenic | 1.28 | mg/L | 0.0100 | EPA-200.7 |
| Barium | 0.0540 | mg/L | 0.0100 | EPA-200.7 |
| Cadmium | <0.0020 | mg/L | 0.0020 | EPA-200.7 |
| Lead | 0.0120 | mg/L | 0.0060 | EPA-200.7 |
| Mercury | <0.00020 | mg/L | 0.00020 | EPA-245.1 |
| Selenium | <0.0100 | mg/L | 0.000 | EPA-200.7 |
| Silver | <0.0020 | mg/L | 0.0020 | EPA-200.7 |

TABLE 2

| | | | Method | |
|---|---|---|---|---|
| | | | Quantification | Analytical |
| Test | Results | Units | Limit | Method |
| Fluoride (w/o | 1.12 | mg/L | 0.125 | EPA-300 |
| distillation) | | | | |
| Nitrate (NO3—N) | 2.47 | mg/L | 0.100 | EPA-300 |
| Total Dissolved Solids | 380 | mg/L | 50.5 | 2540C-2011 |
| Arsenic | 0.0583 | mg/L | 0.0100 | EPA-200.7 |
| Barium | 0.0310 | mg/L | 0.0100 | EPA-200.7 |
| Cadmium | <0.0020 | mg/L | 0.0020 | EPA-200.7 |
| Lead | <0.0060 | mg/L | 0.0060 | EPA-200.7 |
| Mercury | <0.00020 | mg/L | 0.00020 | EPA-245.1 |
| Selenium | <0.0100 | mg/L | 0.000 | EPA-200.7 |
| Silver | <0.0020 | mg/L | 0.0020 | EPA-200.7 |

### Plasmonics

The copper and other metal hydroxides of the embodiments are useful in plasmonics-based devices. Plasmonics takes advantage of the coupling of light to charges like electrons in metals, and allows breaking the diffraction limit for the localization of light into subwavelength dimensions enabling strong field enhancements. Plasmonic nanoparticles are discrete metallic particles that have unique optical properties due to their size and shape, and are increasingly being incorporated into commercial products and technologies. These technologies, which span fields ranging from photovoltaics to biological and chemical sensors, take advantage of the extraordinary efficiency of plasmonic nanoparticles at absorbing and scattering light. Additionally, unlike most dyes and pigments, plasmonic nanoparticles have a color that depends on their size and shape and can be tuned to optimize performance for individual applications without changing the chemical composition of the material.

With respect to the copper hydroxide nanoplatelets, the platelets are flat, reducing the significance of ripple as light passes from one particle to another. By selecting the core material as a base platelet, waveguide and related properties can be modified. By selecting the exterior shell metal, allowable frequency and related properties can be modified. Accordingly, a nanoparticle with a particular combination of core and shell (e.g., magnesium hydroxide core with copper surface layer) will yield a product with desired waveguide properties at a desired frequency. The methods of the embodiments have additional benefits in terms of process conditions (production at room temperature and ambient pressure) and the ability to produce large volumes at low cost.

Plasmon-carrying nanoplatelets of the preferred embodiment can be incorporated into various devices, including, but not limited to, microscopes, light-emitting diodes (LEDs), as well as chemical and biological sensors. Plasmons of the preferred embodiment can also be incorporated into data-carrying integrated circuits with electrical interconnects.

In one embodiment, the nanoplatelets of the preferred embodiments are incorporated into a plasmonic device through placement in a reducing environment with reducing gas. The top layer of the particular metal hydroxide can be converted to its elemental form, encapsulating the underlying hydroxide or oxide material, a dielectric material, of that base nanoplatelet.

As discussed above, nanoplatelets of metal hydroxide are mixed into a water column or other suitable fluid as a precursor to form the dielectric core of a monolayer nanoplatelet, whereas metal salts or other ion sources to supply the ions that are dissolved into the water column or other suitable fluid to supply the ions to self assemble, by ion exchange, the monolayer shell, thereby creating a metal hydroxide monolayer nanoplatelets. Such nanoplatelets can be converted into a plasmonics device through placement in a reducing environment with a reducing gas. The top layer of the metal hydroxide will be converted to its elemental form, encapsulating the underlying hydroxide or oxide material of the core, which is a dielectric material.

The metal hydroxide nanoplatelet's core and other metal hydroxides in the reduced shell, as described in the embodiments, are useful in plasmonic-based devices. Plasmonics takes advantage of the coupling of light to charges like electrons in metals and allows breaking the diffraction limit for the localization of light into subwavelength dimensions enabling strong field enhancements. Plasmonic nanoparticles are discrete metallic particles that have unique optical properties and in the electromagnet spectrum, due to their size and shape, and are increasingly being incorporated into commercial products and technologies. These technologies, which span fields ranging from photovoltaics to biological and chemical sensors, take advantage of the extraordinary efficiency of plasmonic nanoparticles at absorbing and scattering light. Additionally, unlike most dyes and pigments, plasmonic nanoparticles have a color that depends on their size and shape and can be tuned to optimize performance for individual applications without changing the chemical composition of the material.

With respect to the monolayer hydroxide nanoplatelets, the platelets are flat, reducing the significance of ripple as light passes from one particle to another. By selecting the core material as a base platelet, waveguide and related properties can be modified. By selecting the exterior shell metal, allowable frequency and related properties can be modified. Accordingly, a nanoparticle with a particular combination of core and shell (e.g., magnesium hydroxide core with copper layer) will yield a product with desired waveguide properties at a desired frequency. The methods of the embodiments have additional benefits in terms of process conditions (production at room temperature and ambient pressure) and the ability to produce large volumes at low cost.

Plasmon-carrying nanoplatelets of the preferred embodiment can be incorporated into various devices, including, but not limited to, microscopes, light-emitting diodes (LEDs), as well as chemical and biological sensors. Plasmons of the embodiments can also be incorporated into data-carrying integrated circuits with electrical interconnects.

### Antimicrobial Activity—Pressure Treated Lumber

The copper hydroxide nanoparticles prepared by the methods as described herein are useful for preparing pressure treated lumber with resistance to fungus and other microbes. Copper hydroxide nanoparticles of a small size (e.g., dimensions in the X direction of 30 nm, the Y direction of 30 nm, and the Z direction of 0.7 nm) up to a larger size (e.g., dimensions in the X direction of 150 nm, the Y direction of 150 nm, and the Z direction of 10 nm) can be produced that yield superior penetration and distribution of the copper to the lumber's vascular system than is observed for micronized copper as is conventionally employed in the industry. The copper hydroxide nanoparticles provide a single component efficacious treatment for use in preparing pressure treated lumber that is resistant to rot and which exhibits fungicidal properties that extend into years of protection. It is also far less toxic the current products conventionally employed in the industry. The methodology is amenable to large volume production capability and low cost production.

The methodology can also be employed to impart a zinc, titanium, or other antimicrobial element as an outer shell of a magnesium hydroxide nanoparticle, so as to deliver the element into the lumber's vasculature.

In certain embodiments, the metal (e.g., copper) hydroxide monolayer nanoparticles of a small size (e.g., dimensions in the X direction of 30 nm, the Y direction of 30 nm, and the Z direction of 1 nm) up to a larger size (e.g., dimensions in the X direction of 150 nm, the Y direction of 150 nm, and the Z direction of 10 nm) can be produced that yield superior penetration and distribution of the copper monolayer nanoparticles to the lumber's vascular system. The nanoplatelets can further comprise a shell encasing a core, wherein the shell comprises copper hydroxide and the core comprises magnesium hydroxide. The nanoplatelets can further comprise a shell encasing a core, wherein the shell comprises zinc hydroxide and the core comprises magnesium hydroxide. The nanoplatelets can further comprise a shell encasing a core, wherein the shell comprises titanium hydroxide and the core comprises magnesium hydroxide. The nanoplatelets can further comprise a shell encasing a core, wherein the shell comprises copper, zinc, and/or titanium hydroxide and the core comprises magnesium hydroxide.

The methodology can also be employed to impart a zinc, titanium, or other antimicrobial element as an outer shell of a magnesium hydroxide monolayer nanoparticle (or other metal core nanoparticle), to deliver the antimicrobial element(s) into the lumber's vasculature system. For example, the nanoplatelets can further comprise a partial shell encasing a core having the molar content of the outer layer of the core to stoichiometric balanced molar content of the ions to produce a shell from about 1% to 99% coverage of the core with shell metal ions or mixed metal ions and the core comprising magnesium hydroxide. In an embodiment, the nanoplatelets comprise individual crystallites.

### Mining of Metal

Metal hydroxide as described above can be employed in certain types of mining. The nanoparticulate form comprises a core of a monolayer (or thicker) metal hydroxide, wherein the metal hydroxide is more reactive than the metal sought to be recovered. The outer most layer of the core metal hydroxide is exposed to a fluid, e.g., an aqueous or other suitable fluid, for ion exchange and containing a dissolved ion species not as reactive and not the same as the core metal species. This reactive metal forms a shell on the monolayer hydroxide, and the methodology can be employed to scavenge valuable ions (e.g., of rare earth metals or noble metals). The resulting metal monolayer nanoplatelets can then be subjected to, e.g., electrowinning or thermal reduction to secure the metal in a purified form. Such a process can be employed in mining of raw materials or in other resource extraction processes, e.g., recovery of metals from recycled electronics or other metal sources.

The monolayer shell can be formed by ion exchange from the hydroxide core with a less reactive metal ion from the water column or other suitable fluid there by reducing that shell species concentration in the fluid column and increasing core ion content of the fluid column. Metal hydroxide monolayer nanoplatelets can be employed with the core comprised of a more reactive metal hydroxide, and a less reactive metal hydroxide shell, wherein the metal of the shell is not same as the metal of the metal hydroxide core, having platelet diameter of from about 30 nm to about 3500 nm and thickness of from about 1 nm to about 400 nm., and comprising individual crystallites.

Once the ion exchange process has been completed, one can recover the sequestered metal and convert it to a more valuable form, e.g., a metallic form or alternative ionic form. For example, in an alternative method, magnesium hydroxide nanoparticles can be employed to scavenge valuable ions (e.g., of rare earth metals or noble metals). The resulting metal monolayer nanoplatelets can then be subjected to, e.g., electrowinning or thermal reduction to secure the metal in a purified form. Such a process can be employed in mining of raw materials or in other resource extraction processes, e.g., recovery of metals from recycled electronics or other metal sources.

While hydroxide monolayer nanoparticles (or less reactive hydroxide shell formed on a more reactive hydroxide nanoparticles core) can be advantageously prepared using the ion exchange method described above, the method can also be used to prepare selective metal hydroxide monolayer nanoparticles. In such cases, magnesium ion is more labile than the other metal with respect to association with hydroxide. Other more labile ions can alternatively be employed to recover a less labile metal. Other metals that can be employed in ion exchange include the rare earth elements and transition metals (including the noble metals). Transition metals include scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, gold, mercury, rutherfordium, dubnium, seaborgium, bohrium, hassium, meitnerium, ununnilium, ununennium, and ununbium. Rare earth elements include the lanthanide series (lanthanum, cerium, praseodymium, neodymium, promethium, samarium, europium, gadolinium, terbium, dysprosium, holmium, erbium, thulium, ytterbium, and lutetium) and the actinide series (actinium, thorium, protactinium, uranium, neptunium, plutonium, americium, curium, berkelium, californium, einsteinium, fermium, mendelevium, nobelium, and lawrencium). The resulting nanoparticles may advantageously be employed for various uses as a valuable material in their own right.

Such nanoplatelets can have a dimension in the X axis of from about 30 nm to about 3500 nm, a dimension in the Y axis of about 30 nm to about 3500 nm, and a dimension in the Z axis of from 1 nm to 400 nm, wherein the nanoplatelet comprises a more reactive hydroxide core and a less reactive metal hydroxide or rare earth hydroxide shell, wherein the metal or rare earth is not same element as the metal in the core.

The core of the monolayer metal hydroxide in certain embodiments is as small as possible, such that the shell formed by the harvest of metal ion is maximized, thereby producing a richer ore by weight.

In other embodiments, nanoplatelets are provided having a dimension in the X axis of from about 30 nm to about 100 nm, a dimension in the Y axis of about 30 nm to about 100 nm, and a dimension in the Z axis of from 1 nm to 10 nm, wherein the nanoplatelet comprises a more reactive hydroxide core and a less reactive metal hydroxide or rare earth hydroxide shell, wherein the metal or rare earth is not same element as the core to form monolayer nanoparticles.

The nanoplatelet's shell metal can be a transition metal selected from the group consisting of scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, gold, mercury, rutherfordium, dubnium, seaborgium, bohrium, hassium, meitnerium, ununnilium, ununennium, and ununbium.

The nanoplatelet's shell metal can be a lanthanide series element selected from the group consisting of lanthanum, cerium, praseodymium, neodymium, promethium, samarium, europium, gadolinium, terbium, dysprosium, holmium, erbium, thulium, ytterbium, and lutetium.

The nanoplatelet's shell metal can be an actinide series element selected from the group consisting of actinium, thorium, protactinium, uranium, neptunium, plutonium, americium, curium, berkelium, californium, einsteinium, fermium, mendelevium, nobelium, and lawrencium.

The shell can comprise a monolayer of the metal or rare earth hydroxide, e.g., where the nanoplatelets comprise individual crystallites.

Methods and devices that are suitable for use in conjunction with aspects of the preferred embodiments are disclosed in: U.S. Pat. No. 5,264,097, entitled “ELECTRODIALYTIC CONVERSION OF COMPLEXRES AND SALTS OF METAL CATIONS,” U.S. Pat. No. 3,959,095; entitled “METHODS OF OPERATING A THREE COMPARTMENT ELECTROLYTIC CELL FOR THE PRODUCTION OF ALKALLI METAL HYDROXIDES;” U.S. Publication No. 20070022839, entitled “SYNTHESES AND APPLICATIONS OF NANO-SIZED IRON PARTICLES;” U.S. Pat. No. 7,172,747, entitled “METAL OXIDE NANOTUBE AND PROCESS FOR PRODUCTION THEREOF;” U.S. Pat. No. 6,656,339, entitled “METHOD OF FORMING A NANO-SUPPORTED CATALYST ON A SUBSTRATE FOR NANOTUBE GROWTH;” U.S. Pat. No. 5,470,910, entitled “COMPOSITE MATERIALS CONTAINING NANOSCALAR PARTICLES, PROCESS FOR PRODUCING THEM AND THEIR USE FOR OPTICAL COMPONENTS;” U.S. Publication No. 20070098806, entitled “POLYMER-BASED ANTIMICROBIAL AGENTS, METHODS OF MAKING SAID AGENTS, AND PRODUCTS INCORPORATING SAID AGENTS;” U.S. Publication No. 20060216602, entitled “MACROSCOPIC ASSEMBLY OF NANOMETRIC FILAMENTARY STRUCTURES AND METHOD OF PREPARATION THEREOF;” U.S. Publication No. 20060193766, entitled “TITANIA NANOTUBE AND METHOD FOR PRODUCING SAME;” U.S. Publication No. 20060159603, entitled “SEPARATION OF METAL NANOPARTICLES;” Boo et al., Fracture Behaviour of Nanoplatelet Reinforced Polymer Nanocomposites, Mat. Sci. and Tech. 22(7) 2006: 829-834; Li et al., Structure and Magnetic Properties of Cobalt Nanoplatelets, Mat. Lett. 58 (2004): 2506-2509; Zhou et al., Preparation and Characterization of Nanoplatelets of Nickel Hydroxide and Nickel Oxide, Mat. Chem. and Phys. 98(2006): 267-272; Sun et al., From Layered Double Hydroxide to Spinel Nanostructures: Facile Synthesis and Characterization of Nanoplatelets and Nanorods, J. Phys. Chem. B. 110 (2006): 13375-13380; Zarate et al., Novel Route to Synthesize CuO Nanoplatelets, J. Sol. St. Chem. 180(2007): 1464-1469; Shouzhu et al., Nanofibers and Nanoplatelets of MoO₃ via an Electrospinning Technique, J. Phys. and Chem. Of Sol. 67(2006): 1869-1872; Hou et al., High-Yield Preparation of Uniform Cobalt Hydroxide and Oxide Nanoplatelets and Their Characterization, J. Phys. Chem. B. 109(2005): 19094-19098; Liu et al., Facile and Large-Scale Production of ZnO/Zn—Al Layered Double Hydroxide Hierarchical Heterostructures, J. Phys. Chem B. 110(2006): 21865-21872; “Light is a wonderful medium for carrying information”, Scientific American, pp. 58-63, April 2007.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, and phosphates.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, tocopherols, carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood. When a tincture or other liquid form is prepared, animal, vegetable oils, or mineral oils suitable for human consumption can advantageously be employed as diluents. For example, suitable vegetable oils include but are not limited to olive oil, coconut oil, MCT (mixed chain triglycerides derived from coconut oil), and avocado oil.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles, and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

As used in the claims below and throughout this disclosure, by the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method of removing ions of a metal other than magnesium, or rare earth ions, from a substance, comprising:
exposing the substance to magnesium hydroxide nanoplatelets, whereby magnesium ions are replaced by the metal ions or the rare earth ions, yielding a nanoplatelet comprising a hydroxide of the metal or a hydroxide of the rare earth; and
sequestering the nanoplatelets comprising the hydroxide of the metal or the hydroxide of the rare earth from the substance;
wherein the metal ions or the rare earth ions self-assemble by ion exchange to yield a monolayer shell, thereby creating a metal hydroxide monolayer nanoplatelet.

2. The method of claim 1, wherein the substance is water that has been exposed to a fly ash or mine tailings.

3. The method of claim 1, wherein the method is a method of mining the metal or the rare earth.

4. The method of claim 1, wherein the resulting nanoplatelets having a metal hydroxide monolayer are further subjected to electrowinning or thermal reduction to recover the sequestered metal ions.

5. The method of claim 1, wherein the metal hydroxide monolayer nanoplatelet comprises a core comprised of magnesium hydroxide, and a metal hydroxide shell, wherein the shell does not comprise magnesium, the nanoplatelet having a platelet diameter of from about 30 nm to about 3500 nm, a thickness of from about 1 nm to about 400 nm and an aspect ratio of from 15 to 75.

6. The method of claim 1, wherein the metal hydroxide monolayer nanoplatelet comprises an individual crystallite.

7. The method of claim 1, wherein the ions of a metal other than magnesium comprise a lanthanide series elements, ions selected from the group consisting of lanthanum, cerium, praseodymium, neodymium, promethium, samarium, europium, gadolinium, terbium, dysprosium, holmium, erbium, thulium, ytterbium, and lutetium individually or mixtures thereof, and the core comprises magnesium hydroxide.

8. The method of claim 1, wherein the ions of a metal other than magnesium comprise a transition metal ions selected from the group consisting of scandium, titanium, vanadium, chromium, manganese, iron, cobalt, nickel, zinc, yttrium, zirconium, niobium, molybdenum, technetium, ruthenium, rhodium, palladium, silver, cadmium, hafnium, tantalum, tungsten, rhenium, osmium iridium, platinum, copper, gold, mercury, rutherfordium, dubnium, seaborgium, bohrium, hassium, meitnerium, ununnilium, ununennium, and ununbium, individually or mixtures thereof, and the core comprises magnesium hydroxide.

9. The method of claim 1, wherein rare earth ions comprise an actinide series element, ions selected from the group consisting of actinium, thorium, protactinium, uranium, neptunium, plutonium, americium, curium, berkelium, californium, einsteinium, fermium, mendelevium, nobelium, and lawrencium, individually or mixtures thereof, and the core comprises magnesium hydroxide.

10. The method of claim 1, wherein the ions of a metal other than magnesium comprise metal ions selected from the group consisting of titanium, zinc, silver and copper, individually or mixtures thereof, and the core comprises magnesium hydroxide.

11. The method of claim 1, wherein the molar content of the outer layer of the core to a stoichiometric balanced molar content of the ions to produce a shell providing from about 1% to 99% coverage of the core with individual metal ions or mixed metal ions.
